SEQUENCE LISTING (28 sequences)

SEQ ID NO: 1 - protein, 5 aa, Artificial Sequence
  source: /note=“Description of Artificial Sequence Synthetic peptide”
NYWMH

SEQ ID NO: 2 - protein, 17 aa, Artificial Sequence
  source: /note=“Description of Artificial Sequence Synthetic peptide”
ATYRGHSDTYYNQKFKG

SEQ ID NO: 3 - protein, 12 aa, Artificial Sequence
  source: /note=“Description of Artificial Sequence Synthetic peptide”
GAIYDGYDVLDN

SEQ ID NO: 4 - protein, 11 aa, Artificial Sequence
  source: /note=“Description of Artificial Sequence Synthetic peptide”
SASQDISNYLN

SEQ ID NO: 5 - protein, 7 aa, Artificial Sequence
  source: /note=“Description of Artificial Sequence Synthetic peptide”
YTSNLHS

SEQ ID NO: 6 - protein, 9 aa, Artificial Sequence
  source: /note=“Description of Artificial Sequence Synthetic peptide”
QQYRKLPWT

SEQ ID NO: 7 - protein, 121 aa, Artificial Sequence
  source: /note=“Description of Artificial Sequence Synthetic polypeptide”
QVQLVQSGAEVKKPGSSVKVSCKASGGTFSNYWMHWVRQAPGQGLEWMGATYRGHSDTYYNQKFKGRVTITADKSTSTAYMELSSLRSEDTAVYYCARGAIYDGYDVLDNWGQGTLVTVSS

SEQ ID NO: 8 - protein, 108 aa, Artificial Sequence
  source: /note=“Description of Artificial Sequence Synthetic polypeptide”
DIQMTQSPSSLSASVGDRVTITCSASQDISNYLNWYQQKPGKAPKLLIYYTSNLHSGVPSRFSGSGSGTDFTLTISSLQPEDFATYYCQQYRKLPWTFGQGTKLEIKR

SEQ ID NO: 9 - protein, 451 aa, Artificial Sequence
  source: /note=“Description of Artificial Sequence Synthetic polypeptide”
QVQLVQSGAEVKKPGSSVKVSCKASGGTFSNYWMHWVRQAPGQGLEWMGATYRGHSDTYYNQKFKGRVTITADKSTSTAYMELSSLRSEDTAVYYCARGAIYDGYDVLDNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 214 aa, Artificial Sequence
  source: /note=“Description of Artificial Sequence Synthetic polypeptide”
DIQMTQSPSSLSASVGDRVTITCSASQDISNYLNWYQQKPGKAPKLLIYYTSNLHSGVPSRFSGSGSGTDFTLTISSLQPEDFATYYCQQYRKLPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 168 aa, Homo sapiens
MKSGLWYFFLFCLRIKVLTGEINGSANYEMFIFHNGGVQILCKYPDIVQQFKMQLLKGGQILCDLTKTKGSGNTVSIKSLKFCHSQLSNNSVSFFLYNLDHSHANYYFCNLSIFDPPPFKVTLTGGYLHIYESQLCCQLKFWLPIGCAAFVVVCILGCILICWLTKKM

SEQ ID NO: 12 - protein, 199 aa, Homo sapiens
MKSGLWYFFLFCLRIKVLTGEINGSANYEMFIFHNGGVQILCKYPDIVQQFKMQLLKGGQILCDLTKTKGSGNTVSIKSLKFCHSQLSNNSVSFFLYNLDHSHANYYFCNLSIFDPPPFKVTLTGGYLHIYESQLCCQLKFWLPIGCAAFVVVCILGCILICWLTKKKYSSSVHDPNGEYMFMRAVNTAKKSRLTDVTL

SEQ ID NO: 13 - protein, 5 aa, Artificial Sequence
  source: /note=“Description of Artificial Sequence Synthetic peptide”
DYAMH

SEQ ID NO: 14 - protein, 17 aa, Artificial Sequence
  source: /note=“Description of Artificial Sequence Synthetic peptide”
LISIYSDHTNYNQKFQG

SEQ ID NO: 15 - protein, 12 aa, Artificial Sequence
  source: /note=“Description of Artificial Sequence Synthetic peptide”
NNYGNYGWYFDV

SEQ ID NO: 16 - protein, 10 aa, Artificial Sequence
  source: /note=“Description of Artificial Sequence Synthetic peptide”
SASSSVSYMH

SEQ ID NO: 17 - protein, 7 aa, Artificial Sequence
  source: /note=“Description of Artificial Sequence Synthetic peptide”
DTSKLAS

SEQ ID NO: 18 - protein, 9 aa, Artificial Sequence
  source: /note=“Description of Artificial Sequence Synthetic peptide”
FQGSGYPYT

SEQ ID NO: 19 - protein, 121 aa, Artificial Sequence
  source: /note=“Description of Artificial Sequence Synthetic polypeptide”
QVQLVQSGAEVKKPGSSVKVSCKASGYTFTDYAMHWVRQAPGQGLEWMGLISIYSDHTNYNQKFQGRVTITADKSTSTAYMELSSLRSEDTAVYYCGRNNYGNYGWYFDVWGQGTTVTVSS

SEQ ID NO: 20 - protein, 106 aa, Artificial Sequence
  source: /note=“Description of Artificial Sequence Synthetic polypeptide”
EIVLTQSPATLSLSPGERATLSCSASSSVSYMHWYQQKPGQAPRLLIYDTSKLASGIPARFSGSGSGTDYTLTISSLEPEDFAVYYCFQGSGYPYTFGQGTKLEIK

SEQ ID NO: 21 - protein, 116 aa, Artificial Sequence
  source: /note=“Description of Artificial Sequence Synthetic polypeptide”
EVQLVESGGLVQPGGSLRLSCAASGFTFSDYWMDWVRQAPGKGLVWVSNIDEDGSITEYSPFVKGRFTISRDNAKNTLYLQMNSLRAEDTAVYYCTRWGRFGFDSWGQGTLVTVSS

SEQ ID NO: 22 - protein, 111 aa, Artificial Sequence
  source: /note=“Description of Artificial Sequence Synthetic polypeptide”
DIVMTQSPDSLAVSLGERATINCKSSQSLLSGSFNYLTWYQQKPGQPPKLLIFYASTRHTGVPDRFSGSGSGTDFTLTISSLQAEDVAVYYCHHHYNAPPTFGPGTKVDIK

SEQ ID NO: 23 - protein, 10 aa, Artificial Sequence
  source: /note=“Description of Artificial Sequence Synthetic peptide”
GFTFSDYWMD

SEQ ID NO: 24 - protein, 17 aa, Artificial Sequence
  source: /note=“Description of Artificial Sequence Synthetic peptide”
NIDEDGSITEYSPFVKG

SEQ ID NO: 25 - protein, 8 aa, Artificial Sequence
  source: /note=“Description of Artificial Sequence Synthetic peptide”
WGRFGFDS

SEQ ID NO: 26 - protein, 15 aa, Artificial Sequence
  source: /note=“Description of Artificial Sequence Synthetic peptide”
KSSQSLLSGSFNYLT

SEQ ID NO: 27 - protein, 7 aa, Artificial Sequence
  source: /note=“Description of Artificial Sequence Synthetic peptide”
YASTRHT

SEQ ID NO: 28 - protein, 9 aa, Artificial Sequence
  source: /note=“Description of Artificial Sequence Synthetic peptide”
HHHYNAPPT

(19) United States
(12) Patent Application Publication
(10) Pub. No.: US 2020/0255526 A1
(43) Pub. Date: Aug. 13, 2020

(54) COMBINATION TREATMENT FOR CANCER

(71) Applicant: GLAXOSMITHKLINE INTELLECTUAL PROPERTY DEVELOPMENT LIMITED, BRENTFORD (GB)

(72) Inventors: Axel HOOS, Collegeville, PA (US); Sanjay KHANDEKAR, Collegeville, PA (US); Patrick MAYES, Devon, PA (US); Joanna OPALINSKA, Collegeville, PA (US)

(21) Appl. No.: 16/646,921

(22) PCT Filed: Sep. 12, 2018
(86) PCT No.: PCT/IB2018/056969

§ 371 Date: Mar. 12, 2020

Related U.S. Application Data
(60) Provisional application No. 62/558,608, filed on Sep. 14, 2017.

(51) Int. Cl. C07K 16/28; A61K 47/68; A61P 43/00

(52) U.S. Cl. CPC C07K 16/2818; A61K 47/6803; A61K 47/6849; A61K 2039/507; C07K 16/2878; A61P 43/00; C07K 16/2896

(57) ABSTRACT

Disclosed herein is a method of treating cancer, such as multiple myeloma, involving the combination of an anti-BCMA antigen binding protein (e.g., an anti-BCMA antibody) and an immunomodulatory agent (e.g. an agent directed to ICOS or an anti-CD38 antigen binding protein).

### SEQUENCE LISTING

The instant application contains a Sequence Listing which has been submitted electronically in ASCII format and is hereby incorporated by reference in its entirety. Said ASCII copy, created on Sep. 10, 2018, is named PU66430_WO_SL.txt and is 21,096 bytes in size.

### FIELD OF THE INVENTION

The present invention relates to methods of treating cancer in a subject. In particular, the present invention relates to a combination of an anti-BCMA antigen binding protein and an immunomodulatory agent for treating cancer.

### BACKGROUND TO THE INVENTION

Multiple myeloma (MM) is an incurable malignancy and accounts for 1% of all cancers and for 10% of all hematologic malignancies. A variety of drugs and combination treatments have been evaluated and found effective in treating multiple myeloma (National Comprehensive Cancer Network, 2016; Moreau, San Miguel et al., 2017). However, most, if not all, of these patients inevitably relapse (Richardson, Barlogie et al., 2003; Richardson, Barlogie et al., 2006; Jagannath, Barlogie et al., 2008).

Three and four-drug combinations are emerging for patients with previously treated MM but these regimens may be limited by toxic effects (National Comprehensive Cancer Network, 2016). Agents with new mechanisms of action that can be combined with existing therapies without an increase in serious toxicity are needed. Therefore, there is an urgent need to develop treatment combinations with mechanism of action that do not overlap, and where cross-resistance with prior treatments could be minimized.

### SUMMARY OF THE INVENTION

The disclosure relates to methods of treating cancer in a subject, e.g. a human. In particular, the present invention relates to a combination of an anti-BCMA antigen binding protein, such as an antibody, and an immunomodulatory agent for treating cancer. In one embodiment, the cancer is selected from multiple myeloma, chronic lymphocytic leukemia, and non-Hodgkin's lymphoma.

Provided herein is a method of treating cancer in a subject in need thereof comprising administering a therapeutically effective dose of a combination comprising an anti-BCMA antigen binding protein and an immunomodulatory agent. In one embodiment, the immunomodulatory agent is an agent directed to ICOS, such as an anti-ICOS antibody. In yet another aspect, the anti-ICOS antibody is an ICOS agonist.

In another embodiment, the immunomodulatory agent is an anti-CD38 antigen binding protein, such as daratumumab.

Also provided herein is a method of treating cancer in a subject in need thereof comprising administering a therapeutically effective dose of a combination comprising an anti-BCMA antigen binding protein and an immunomodulatory agent wherein the antibody comprises a CDRH1 comprising an amino acid sequence with at least 90% sequence identity to the amino acid sequence set forth in SEQ ID NO:1; a CDRH2 comprising an amino acid sequence with at least 90% sequence identity to the amino acid sequence set forth in SEQ ID NO:2; a CDRH3 comprising an amino acid sequence with at least 90% sequence identity to the amino acid sequence set forth in SEQ ID NO:3; a CDRL1 comprising an amino acid sequence with at least 90% sequence identity to the amino acid sequence set forth in SEQ ID NO:4; a CDRL2 comprising an amino acid sequence with at least 90% sequence identity to the amino acid sequence set forth in SEQ ID NO:5; and a CDRL3 comprising an amino acid sequence with at least 90% sequence identity to the amino acid sequence set forth in SEQ ID NO:6.

Further provided herein is a method of treating cancer in a subject in need thereof comprising administering a therapeutically effective dose of a combination comprising an anti-BCMA antigen binding protein and an immunomodulatory agent, wherein the anti-BCMA antigen binding protein is an antibody comprising a VH comprising an amino acid sequence with at least 90% sequence identity to the amino acid sequence set forth in SEQ ID NO:7; and a VL comprising an amino acid sequence with at least 90% sequence identity to the amino acid sequence set forth in SEQ ID NO:8.

Further provided herein is a method of treating cancer in a subject in need thereof comprising administering a therapeutically effective dose of a combination comprising an anti-BCMA antigen binding protein and an immunomodulatory agent, wherein the anti-BCMA antigen binding protein is an immunoconjugate comprising an antibody conjugated to a cytotoxin. In one embodiment, the cytotoxin is MMAE or MMAF.

Further provided herein is a method of treating cancer in a subject in need thereof comprising administering a therapeutically effective dose of a combination comprising an anti-BCMA antigen binding protein and an anti-ICOS antibody, wherein the anti-ICOS antibody comprises a CDRH1 comprising an amino acid sequence with at least 90% sequence identity to the amino acid sequence set forth in SEQ ID NO:13; a CDRH2 comprising an amino acid sequence with at least 90% sequence identity to the amino acid sequence set forth in SEQ ID NO:14; a CDRH3 comprising an amino acid sequence with at least 90% sequence identity to the amino acid sequence set forth in SEQ ID NO:15; a CDRL1 comprising an amino acid sequence with at least 90% sequence identity to the amino acid sequence set forth in SEQ ID NO:16; a CDRL2 comprising an amino acid sequence with at least 90% sequence identity to the amino acid sequence set forth in SEQ ID NO:17; and a CDRL3 comprising an amino acid sequence with at least 90% sequence identity to the amino acid sequence set forth in SEQ ID NO:18.

Further provided herein is a method of treating cancer in a subject in need thereof comprising administering a therapeutically effective dose of a combination comprising an anti-BCMA antigen binding protein and an anti-ICOS antibody, wherein the anti-ICOS antibody comprises a a VH domain comprising an amino acid sequence at least 90% identical to the amino acid sequence set forth in SEQ ID NO:19; and a VL domain comprising an amino acid sequence at least 90% identical to the amino acid sequence as set forth in SEQ ID NO:20.

In one embodiment, the agent directed to ICOS comprises an Fc region comprising a S228P mutation and L235E mutation.

Also provided is a combination for use in the treatment of cancer, wherein the combination comprises an anti-BCMA antigen binding protein and an agent directed to ICOS.

Further provided is a combination for use in the treatment of cancer, wherein the combination comprises an anti-BCMA antigen binding protein and an anti-CD38 antigen binding protein.

Also provided is use of a combination in the manufacture of a medicament for use in the treatment of cancer, wherein the combination comprises an anti-BCMA antigen binding protein and an agent directed to ICOS.

Further provided is use of a combination in the manufacture of a medicament for use in the treatment of cancer, wherein the combination comprises an anti-BCMA antigen binding protein and an anti-CD38 antigen binding protein.

Also provided is a kit for use in the treatment of cancer comprising:
(i) an anti-BCMA antigen binding protein;
(ii) instructions for use in the treatment of cancer when combined with an anti-ICOS antibody.

Further provided is a kit for use in the treatment of cancer comprising:
(i) an anti-BCMA antigen binding protein;
(ii) instructions for use in the treatment of cancer when combined with an anti-CD38 antigen binding protein.

### BRIEF DESCRIPTION OF THE DRAWINGS

FIG. 1 shows mean tumor volume data on day 7 for groups treated with an anti-BCMA antibody drug conjugate in combination with an agent directed to ICOS.

FIG. 2 shows individual tumor volume curves for groups treated with an anti-BCMA antibody drug conjugate in combination with an agent directed to ICOS.

### DETAILED DESCRIPTION OF THE INVENTION

The disclosure relates to methods of treating cancer in a subject. In particular, the present invention relates to a combination of an anti-BCMA antigen binding protein and an immunomodulatory agent for treating cancer. Without being bound by theory, it is believed that the novel combination(s) described herein result in reduced toxicities due to non-overlapping mechanisms of action.

The term “antigen binding protein” as used herein refers to antibodies, antibody fragments and other protein constructs which are capable of binding to the antigen. The antigen binding proteins of the present invention may comprise heavy chain variable regions and light chain variable regions of the invention which may be formatted into the structure of a natural antibody or functional fragment or equivalent thereof. An antigen binding protein of the invention may therefore comprise the VH regions of the invention formatted into a full length antibody, a (Fab′)2 fragment, a Fab fragment, or equivalent thereof (such as scFV, bi- tri- or tetra-bodies, Tandabs etc.), when paired with an appropriate light chain The antibody may be an IgG1, IgG2, IgG3, or IgG4; or IgM; IgA, IgE or IgD or a modified variant thereof The constant domain of the antibody heavy chain may be selected accordingly. The light chain constant domain may be a kappa or lambda constant domain. Furthermore, the antigen binding protein may comprise modifications of all classes e.g. IgG dimers, Fc mutants that no longer bind Fc receptors or mediate Clq binding. The antigen binding protein may also be a chimeric antibody of the type described in WO86/01533 which comprises an antigen binding region and a non-immunoglobulin region. In another aspect the antigen binding protein is selected from the group consisting of a dAb, Fab, Fab′, F(ab′)₂, Fv, diabody, triabody, tetrabody, miniantibody, and a minibody. In one aspect of the present invention the antigen binding protein is a humanised or chimaeric antibody, in a further aspect the antibody is humanised. In one aspect the antibody is a monoclonal antibody.

The term “single variable domain” refers to a folded polypeptide domain comprising sequences characteristic of antibody variable domains. It therefore includes complete antibody variable domains such as VH, VHH and VL and modified antibody variable domains, for example, in which one or more loops have been replaced by sequences which are not characteristic of antibody variable domains, or antibody variable domains which have been truncated or comprise N- or C-terminal extensions, as well as folded fragments of variable domains which retain at least the binding activity and specificity of the full-length domain. A single variable domain is capable of binding an antigen or epitope independently of a different variable region or domain. A “domain antibody” or “dAb(TM)” may be considered the same as a “single variable domain”. A single variable domain may be a human single variable domain, but also includes single variable domains from other species such as rodent nurse shark and Camelid VHH dAbs™. Camelid WEI are immunoglobulin single variable domain polypeptides that are derived from species including camel, llama, alpaca, dromedary, and guanaco, which produce heavy chain antibodies naturally devoid of light chains. Such VHH domains may be humanized according to standard techniques available in the art, and such domains are considered to be “single variable domains”. As used herein VH includes camelid WEI domains.

As used herein the term “agonist” refers to an antigen binding protein including but not limited to an antibody, which upon contact with a co-signalling receptor causes one or more of the following (1) stimulates or activates the receptor, (2) enhances, increases or promotes, induces or prolongs an activity, function or presence of the receptor and/or (3) enhances, increases, promotes or induces the expression of the receptor. Agonist activity can be measured in vitro by various assays know in the art such as, but not limited to, measurement of cell signalling, cell proliferation, immune cell activation markers, cytokine production. Agonist activity can also be measured in vivo by various assays that measure surrogate end points such as, but not limited to the measurement of T cell proliferation or cytokine production.

A “humanized antibody” refers to a type of engineered antibody having its CDRs derived from a non-human donor immunoglobulin, the remaining immunoglobulin-derived parts of the molecule being derived from one or more human immunoglobulin(s). In addition, framework support residues may be altered to preserve binding affinity (see, e.g., Queen et al. Proc. Natl Acad Sci USA, 86:10029-10032 (1989), Hodgson, et al., Bio/Technology, 9:421 (1991)). A suitable human acceptor antibody may be one selected from a conventional database, e.g., the KABAT™ database, Los Alamos database, and Swiss Protein database, by homology to the nucleotide and amino acid sequences of the donor antibody. A human antibody characterized by a homology to the framework regions of the donor antibody (on an amino acid basis) may be suitable to provide a heavy chain constant region and/or a heavy chain variable framework region for insertion of the donor CDRs. A suitable acceptor antibody capable of donating light chain constant or variable framework regions may be selected in a similar manner. It should be noted that the acceptor antibody heavy and light chains are not required to originate from the same acceptor antibody. The prior art describes several ways of producing such humanized antibodies—see, for example, EP-A-0239400 and EP-A-054951.

The term “fully human antibody” includes antibodies having variable and constant regions (if present) derived from human germline immunoglobulin sequences. The human sequence antibodies of the invention may include amino acid residues not encoded by human germline immunoglobulin sequences (e.g., mutations introduced by random or site-specific mutagenesis in vitro or by somatic mutation in vivo). Fully human antibodies comprise amino acid sequences encoded only by polynucleotides that are ultimately of human origin or amino acid sequences that are identical to such sequences. As meant herein, antibodies encoded by human immunoglobulin-encoding DNA inserted into a mouse genome produced in a transgenic mouse are fully human antibodies since they are encoded by DNA that is ultimately of human origin. In this situation, human immunoglobulin-encoding DNA can be rearranged (to encode an antibody) within the mouse, and somatic mutations may also occur. Antibodies encoded by originally human DNA that has undergone such changes in a mouse are fully human antibodies as meant herein. The use of such transgenic mice makes it possible to select fully human antibodies against a human antigen. As is understood in the art, fully human antibodies can be made using phage display technology wherein a human DNA library is inserted in phage for generation of antibodies comprising human germline DNA sequence.

The terms “VH” and “VL” are used herein to refer to the heavy chain variable region and light chain variable region respectively of an antigen binding protein.

“CDRs” are defined as the complementarity determining region amino acid sequences of an antigen binding protein. These are the hypervariable regions of immunoglobulin heavy and light chains There are three heavy chain and three light chain CDRs (or CDR regions) in the variable portion of an immunoglobulin. Thus, “CDRs” as used herein refers to all three heavy chain CDRs, all three light chain CDRs, all heavy and light chain CDRs, or at least two CDRs.

Throughout this specification, amino acid residues in variable domain sequences and full length antibody sequences are numbered according to the Kabat numbering convention. Similarly, the terms “CDR”, “CDRL1”, “CDRL2”, “CDRL3”, “CDRH1”, “CDRH2”, “CDRH3” used in the Examples follow the Kabat numbering convention. For further information, see Kabat et al., Sequences of Proteins of Immunological Interest, 5th Ed., U.S. Department of Health and Human Services, National Institutes of Health (1991).

It will be apparent to those skilled in the art that there are alternative numbering conventions for amino acid residues in variable domain sequences and full length antibody sequences. There are also alternative numbering conventions for CDR sequences, for example those set out in Chothia et al. (1989) Nature 342: 877-883. The structure and protein folding of the antibody may mean that other residues are considered part of the CDR sequence and would be understood to be so by a skilled person.

Other numbering conventions for CDR sequences available to a skilled person include “AbM” (University of Bath) and “contact” (University College London) methods. The minimum overlapping region using at least two of the Kabat, Chothia, AbM and contact methods can be determined to provide the “minimum binding unit”. The minimum binding unit may be a sub-portion of a CDR.

“Percent identity” between a query nucleic acid sequence and a subject nucleic acid sequence is the “Identities” value, expressed as a percentage, that is calculated by the BLASTN algorithm when a subject nucleic acid sequence has 100% query coverage with a query nucleic acid sequence after a pair-wise BLASTN alignment is performed. Such pair-wise BLASTN alignments between a query nucleic acid sequence and a subject nucleic acid sequence are performed by using the default settings of the BLASTN algorithm available on the National Center for Biotechnology Institute's website with the filter for low complexity regions turned off.

“Percent identity” between a query amino acid sequence and a subject amino acid sequence is the “Identities” value, expressed as a percentage, that is calculated by the BLASTP algorithm when a subject amino acid sequence has 100% query coverage with a query amino acid sequence after a pair-wise BLASTP alignment is performed. Such pair-wise BLASTP alignments between a query amino acid sequence and a subject amino acid sequence are performed by using the default settings of the BLASTP algorithm available on the National Center for Biotechnology Institute's website with the filter for low complexity regions turned off.

The query sequence may be 100% identical to the subject sequence, or it may include up to a certain integer number of amino acid or nucleotide alterations as compared to the subject sequence such that the % identity is less than 100%. For example, the query sequence is at least 50, 60, 70, 75, 80, 85, 90, 95, 96, 97, 98, or 99% identical to the subject sequence. Such alterations include at least one amino acid deletion, substitution (including conservative and non-conservative substitution), or insertion, and wherein said alterations may occur at the amino- or carboxy-terminal positions of the query sequence or anywhere between those terminal positions, interspersed either individually among the amino acids or nucleotides in the query sequence or in one or more contiguous groups within the query sequence.

The % identity may be determined across the entire length of the query sequence, including the CDR(s). Alternatively, the % identity may exclude the CDR(s), for example the CDR(s) is 100% identical to the subject sequence and the % identity variation is in the remaining portion of the query sequence, so that the CDR sequence is fixed/intact.

The term “variant” as used herein refers to an amino acid sequence with at least one amino acid variation compared to the reference amino acid sequence and may include, for example, deletions, additions, insertions, translocations, truncations, and/or substitutions.

Chimeric antigen receptors (CARs) have been developed as artificial T cell receptors to generate novel specificities in T cells without the need to bind to MHC-antigenic peptide complexes. These synthetic receptors contain a target binding domain that is associated with one or more signalling domains via a flexible linker in a single fusion molecule. The target binding domain is used to target the T cell to specific targets on the surface of pathologic cells and the signalling domains contain molecular machinery for T cell activation and proliferation. The flexible linker which passes through the T cell membrane (i.e. forming a transmembrane domain) allows for cell membrane display of the target binding domain of the CAR. CARs have successfully allowed T cells to be redirected against antigens expressed at the surface of tumour cells from various malignancies including lymphomas and solid tumours (Jena et al. (2010) Blood, 116(7):1035-44).

The development of CARs has comprised three generations so far. The first generation CARS comprised target binding domains attached to a signalling domain derived from the cytoplasmic region of the CD3zeta or the Fc receptor gamma chains. First generation CARs were shown to successfully redirect T cells to the selected target, however, they failed to provide prolonged expansion and antitumor activity in vivo. The second and third generation CARS have focussed on enhancing modified T cell survival and increasing proliferation by including co-stimulatory molecules, such as CD28, OX-40 (CD134) and 4-1BB (CD137).

T cells bearing CARs could be used to eliminate pathologic cells in a disease setting. One clinical aim would be to transform patient cells with recombinant DNA containing an expression construct for the CAR via a vector (e.g. a lentiviral vector) following aphaeresis and T cell isolation. Following expansion of the T cells they are re-introduced into the patient with the aim of targeting and killing the pathologic target cells.

In one aspect, the transmembrane domain can be derived either from a natural or from a synthetic source. In one aspect, the transmembrane domain can be derived from any membrane-bound or transmembrane protein. Alternatively the transmembrane domain can be synthetic and can comprise predominantly hydrophobic residues such as leucine and valine. For example, the transmembrane domain can be the transmembrane domain of CD proteins, such as CD4, CD8, CD3 or CD28, a subunit of the T cell receptor, such as α, β, γ or δ, a subunit of the IL-2 receptor (a chain), a submit of the Low-Affinity Nerve Growth Factor Receptor (LNGFR or p75) β chain or γ chain), or a subunit chain of Fc receptors.

In one aspect, the transmembrane domain comprises the transmembrane domain of CD4, CD8 or CD28. In a further aspect, the transmembrane domain comprises the transmembrane domain of CD4 or CD8 (e.g. the CD8 alpha chain, as described in NCBI Reference Sequence: NP_001139345.1, incorporated herein by reference). In a yet further aspect, the transmembrane domain comprises the transmembrane domain of CD4.

The intracellular effector domain or “signalling domain” is responsible for intracellular signalling following the binding of the target binding domain to the target. The intracellular effector domain is responsible for the activation of at least one of the normal effector functions of the immune cell in which the CAR is expressed. For example, the effector function of a T cell can be a cytolytic activity or helper activity including the secretion of cytokines. Preferred examples of the effector domain for use in a CAR scaffold can be the cytoplasmic sequences of the natural T cell receptor and co-receptors that act in concert to initiate signal transduction following antigen binding, as well as any derivate or variant of these sequences and any synthetic sequence that has the same functional capability.

Effector domains can be separated into two classes: those that initiate antigen-dependent primary activation, and those that act in an antigen-independent manner to provide a secondary or costimulatory signal. Primary activation effector domains can comprise signalling motifs which are known as immunoreceptor tyrosine-based activation motifs (ITAMs). ITAMs are well defined signalling motifs, commonly found in the intracytoplasmic tail of a variety of receptors, and serve as binding sites for syk/zap70 class tyrosine kinases. Examples of ITAMs used in the invention can include, as non-limiting examples, those derived from CD3zeta, FcRgamma, FcRbeta, FcRepsilon, CD3gamma, CD3delta, CD3epsilon, CDS, CD22, CD79a, CD79b and CD66d. In one aspect, the intracellular effector domain comprises a CD3zeta signalling domain (also known as CD247). Natural TCRs contain a CD3zeta signalling molecule, therefore the use of this effector domain is closest to the TCR construct which occurs in nature.

In one aspect of the invention the intracellular signalling domain is a CD3 zeta effector domain. Effector domains may also provide a secondary or costimulatory signal. T cells additionally comprise costimulatory molecules which bind to cognate costimulatory ligands on antigen presenting cells in order to enhance the T cell response, for example by increasing proliferation activation, differentiation and the like. Therefore, in one aspect, the intracellular effector domain additionally comprises a costimulatory domain. In a further aspect, the costimulatory domain comprises the intracellular domain of a costimulatory molecule, selected from CD28, CD27, 4-1BB (CD137), OX40 (CD134), ICOS (CD278), CD30, CD40, PD-1 (CD279), CD2, CD7, NKG2C (CD94), B7-H3 (CD276) or any combination thereof. In a yet further aspect, the costimulatory domain comprises the intracellular domain of a costimulatory molecule, selected from CD28, CD27, 4-1BB, OX40, ICOS or any combination thereof.

As used herein, the term “effective dose” means that dose of a drug or pharmaceutical agent that will elicit the biological or medical response of a tissue, system, animal or human that is being sought, for instance, by a researcher or clinician. Furthermore, the term “therapeutically effective dose” means any dose which, as compared to a corresponding subject who has not received such dose, results in improved treatment, healing, prevention, or amelioration of a disease, disorder, or side effect, or a decrease in the rate of advancement of a disease or disorder. The term also includes within its scope doses effective to enhance normal physiological function.

### Combinations and Pharmaceutical Compositions

The term “combination” described herein refers to at least two therapeutic agents. As used herein the term “therapeutic agent” is understood to mean a substance that produces a desired effect in a tissue, system, animal, mammal, human, or other subject. In one embodiment the combination is an anti-BCMA antigen binding protein, suitably an anti-BCMA antibody, and at least one additional therapeutic agent. In one embodiment, the combination is an anti-BCMA antigen binding protein and an immunomodulatory agent. In another embodiment, the combination is an anti-BCMA antigen binding protein and an agent directed to ICOS. In yet another embodiment, the combination is an anti-BCMA antigen binding protein and an anti-CD38 antigen binding agent. The combinations described herein are effective in treating cancer.

The administration of the combinations of the invention may be advantageous over the individual therapeutic agents in that the combinations may provide one or more of the following improved properties when compared to the individual administration of a single therapeutic agent alone: i) a greater anticancer effect than the most active single agent, ii) synergistic or highly synergistic anticancer activity, iii) a dosing protocol that provides enhanced anticancer activity with reduced side effect profile, iv) a reduction in the toxic effect profile, v) an increase in the therapeutic window, or vi) an increase in the bioavailability of one or both of the therapeutic agents.

The combinations described herein can be in the form of a pharmaceutical composition. A “pharmaceutical composition” contains a combination described herein, and one or more pharmaceutically acceptable carriers, diluents, or excipients. The carrier(s), diluent(s) or excipient(s) must be acceptable in the sense of being compatible with the other ingredients of the formulation, capable of pharmaceutical formulation, and not deleterious to the recipient thereof.

In one embodiment, each therapeutic agent in a combination is individually formulated into its own pharmaceutical composition and each of the pharmaceutical compositions are administered to treat cancer. In this embodiment, each of the pharmaceutical compositions may have the same or different carriers, diluents or excipients. For example, in one embodiment, a first pharmaceutical composition contains an anti-BCMA antigen binding protein, a second pharmaceutical composition contains an immunomodulatory agent, and the first and second pharmaceutical compositions are both administered to treat cancer.

In one embodiment, each therapeutic agent in a combination is formulated together into a single pharmaceutical composition and administered to treat cancer. For example, in one embodiment, a single pharmaceutical composition contains both an anti-BCMA antigen binding protein and an immunomodulatory agent and is administered as a single pharmaceutical composition to treat cancer.

In one embodiment, the combinations described herein can be further combined with an additional therapeutic agent, e.g., an additional cancer therapeutic agent. The additional therapeutic agent may include, but is not limited to, other immunomodulatory drugs, therapeutic antibodies, CAR-T therapeutics, BiTEs, HDAC inhibitors, proteasome inhibitors (e.g. bortezomib), anti-inflammatory compounds, and immunomodulatory imide drugs (IMiD) (e.g., thalidomide and analogs thereof).

### Anti-BCMA Antigen Binding Proteins

The anti-BCMA antigen binding proteins in the combinations described herein are useful in the treatment or prevention of cancers. Any of the anti-BCMA antigen binding proteins disclosed herein may be used in combination with an immunomodulatory agent for treating cancer. The anti-BCMA antigen binding proteins described herein may bind to human BCMA having, including, for example, human BCMA containing the amino acid sequence of GenBank Accession Number Q02223.2, or genes encoding human BCMA having at least 90 percent homology or at least 90 percent identity thereto.

Exemplary anti-BCMA antigen binding proteins and methods of making the same are disclosed in International Publication No. WO2012/163805 which is incorporated by reference herein in its entirety. Additional exemplary anti-BCMA antigen binding proteins include those described in WO2016/014789, WO2016/090320, WO2016/090327, WO2016/020332, WO2016/079177, WO2014/122143, WO2014/122144, WO2017/021450, WO2016/014565, WO2014/068079, WO2015/166649, WO2015/158671, WO2015/052536, WO2014/140248, WO2013/072415, WO2013/072406, WO2014/089335, US2017/165373, WO2013/154760, and WO2017/051068, each of which is incorporated by reference herein in its entirety.

In one embodiment, the anti-BCMA antigen binding protein has enhanced antibody dependent cell mediated cytotoxic activity (ADCC) effector function. The term “Effector Function” as used herein is meant to refer to one or more of Antibody dependent cell mediated cytotoxic activity (ADCC), Complement-dependent cytotoxic activity (CDC) mediated responses, Fc-mediated phagocytosis and antibody recycling via the FcRn receptor. For IgG antibodies, effector functionalities including ADCC and ADCP are mediated by the interaction of the heavy chain constant region with a family of Fcgamma receptors present on the surface of immune cells. In humans these include FcgammaRI (CD64), FcgammaRII (CD32) and FcgammaRIII (CD16). Interaction between the antigen binding protein bound to antigen and the formation of the Fc/Fcgamma complex induces a range of effects including cytotoxicity, immune cell activation, phagocytosis and release of inflammatory cytokines.

In another embodiment, the anti-BCMA antigen binding proteins described herein inhibit the binding of BAFF and/or APRIL to the BCMA receptor. In another embodiment, the anti-BCMA antigen binding proteins described herein are capable of binding to FcgammaRIIIA or is capable of FcgammaRIIIA mediated effector function.

In one embodiment, the anti-BCMA antigen binding protein is an antibody comprising a heavy chain variable region CDR1 (“CDRH1”) comprising an amino acid sequence with at least 90%, 91%, 92%, 93%, 94%, 95%, 96%, 97%, 98%, 99% or 100% sequence identity to the amino acid sequence set forth in SEQ ID NO:1. In one embodiment, the heavy chain variable region CDR1 (“CDRH1”) comprises an amino acid sequence with one amino acid variation (variant) to the amino acid sequence set forth in SEQ ID NO:1.

In one embodiment, the anti-BCMA antigen binding protein is an antibody comprising a heavy chain variable region CDR2 (“CDRH2”) comprising an amino acid sequence with at least 90%, 91%, 92%, 93%, 94%, 95%, 96%, 97%, 98%, 99% or 100% sequence identity to the amino acid sequence set forth in SEQ ID NO:2. In one embodiment, the heavy chain variable region CDR2 (“CDRH2”) comprises an amino acid sequence with one amino acid variation (variant) to the amino acid sequence set forth in SEQ ID NO:2.

In one embodiment, the anti-BCMA antigen binding protein is an antibody comprising a heavy chain variable region CDR3 (“CDRH3”) comprising an amino acid sequence with at least 90%, 91%, 92%, 93%, 94%, 95%, 96%, 97%, 98%, 99% or 100% sequence identity to the amino acid sequence set forth in SEQ ID NO:3. In one embodiment, the heavy chain variable region CDR3 (“CDRH3”) comprises an amino acid sequence with one amino acid variation (variant) to the amino acid sequence set forth in SEQ ID NO:3.

In one embodiment, the anti-BCMA antigen binding protein is an antibody comprising a light chain variable region CDR1 (“CDRL1”) comprising an amino acid sequence with at least 90%, 91%, 92%, 93%, 94%, 95%, 96%, 97%, 98%, 99% or 100% sequence identity to the amino acid sequence set forth in SEQ ID NO:4. In one embodiment, the light chain variable region CDL1 (“CDR1”) comprises an amino acid sequence with one amino acid variation (variant) to the amino acid sequence set forth in SEQ ID NO:4.

In one embodiment, the anti-BCMA antigen binding protein is an antibody comprising a light chain variable region CDR2 (“CDRL2”) comprising an amino acid sequence with at least 90%, 91%, 92%, 93%, 94%, 95%, 96%, 97%, 98%, 99% or 100% sequence identity to the amino acid sequence set forth in SEQ ID NO:5. In one embodiment, the light chain variable region CDL2 (“CDR2”) comprises an amino acid sequence with one amino acid variation (variant) to the amino acid sequence set forth in SEQ ID NO:5.

In one embodiment, the anti-BCMA antigen binding protein is an antibody comprising a light chain variable region CDR3 (“CDRL3”) comprising an amino acid sequence with at least 90%, 91%, 92%, 93%, 94%, 95%, 96%, 97%, 98%, 99% or 100% sequence identity to the amino acid sequence set forth in SEQ ID NO:6. In one embodiment, the light chain variable region CDL3 (“CDR3”) comprises an amino acid sequence with one amino acid variation (variant) to the amino acid sequence set forth in SEQ ID NO:6.

In one embodiment, the anti-BCMA antigen binding protein is an antibody comprising a CDRH1 comprising an amino acid sequence with at least 90%, 91%, 92%, 93%, 94%, 95%, 96%, 97%, 98%, 99% or 100% sequence identity to the amino acid sequence set forth in SEQ ID NO:1; CDRH2 comprising an amino acid sequence with at least 90%, 91%, 92%, 93%, 94%, 95%, 96%, 97%, 98%, 99% or 100% sequence identity to the amino acid sequence set forth in SEQ ID NO:2; CDRH3 comprising an amino acid sequence with at least 90%, 91%, 92%, 93%, 94%, 95%, 96%, 97%, 98%, 99% or 100% sequence identity to the amino acid sequence set forth in SEQ ID NO:3; CDRL1 comprising an amino acid sequence with at least 90%, 91%, 92%, 93%, 94%, 95%, 96%, 97%, 98%, 99% or 100% sequence identity to the amino acid sequence set forth in SEQ ID NO:4; CDRL2 comprising an amino acid sequence with at least 90%, 91%, 92%, 93%, 94%, 95%, 96%, 97%, 98%, 99% or 100% sequence identity to the amino acid sequence set forth in SEQ ID NO:5; and/or CDRL3 comprising an amino acid sequence with at least 90%, 91%, 92%, 93%, 94%, 95%, 96%, 97%, 98%, 99% or 100% sequence identity to the amino acid sequence set forth in SEQ ID NO:6.

In one embodiment, the anti-BCMA antigen binding protein is an antibody comprising a CDRH1 comprising an amino acid sequence set forth in SEQ ID NO:1; a CDRH2 comprising an amino acid sequence set forth in SEQ ID NO:2; a CDRH3 comprising an amino acid sequence set forth in SEQ ID NO:3; a CDRL1 comprising an amino acid sequence set forth in SEQ ID NO:4; a CDRL2 comprising an amino acid sequence set forth in SEQ ID NO:5; and a CDRL3 comprising an amino acid sequence set forth in SEQ ID NO:6.

In one embodiment, the anti-BCMA antigen binding protein is an antibody comprising a heavy chain variable region (“VH”) comprising an amino acid sequence with at least 90%, 91%, 92%, 93%, 94%, 95%, 96%, 97%, 98%, 99% or 100% sequence identity to the amino acid sequence set forth in SEQ ID NO:7.

In one embodiment, the anti-BCMA antigen binding protein is an antibody comprising a light chain variable region (“VL”) comprising an amino acid sequence with at least 90%, 91%, 92%, 93%, 94%, 95%, 96%, 97%, 98%, 99% or 100% sequence identity to the amino acid sequence set forth in SEQ ID NO:8.

In one embodiment, the anti-BCMA antigen binding protein is an antibody comprising a VH comprising an amino acid sequence with at least 90%, 91%, 92%, 93%, 94%, 95%, 96%, 97%, 98%, 99% or 100% sequence identity to the amino acid sequence set forth in SEQ ID NO:7; and a VL comprising an amino acid sequence with at least 90%, 91%, 92%, 93%, 94%, 95%, 96%, 97%, 98%, 99% or 100% sequence identity to the amino acid sequence set forth in SEQ ID NO:8.

In one embodiment, the anti-BCMA antigen binding protein is an antibody comprising a VH comprising an amino acid sequence set forth in SEQ ID NO:7; and a VL comprising an amino acid sequence set forth in SEQ ID NO:8.

In one embodiment, the anti-BCMA antigen binding protein is an antibody comprising a heavy chain region (“HC”) comprising an amino acid sequence with at least 90%, 91%, 92%, 93%, 94%, 95%, 96%, 97%, 98%, 99% or 100% sequence identity to the amino acid sequence set forth in SEQ ID NO:9.

In one embodiment, the anti-BCMA antigen binding protein is an antibody comprising a a light chain region (“LC”) comprising an amino acid sequence with at least 90%, 91%, 92%, 93%, 94%, 95%, 96%, 97%, 98%, 99% or 100% sequence identity to the amino acid sequence set forth in SEQ ID NO:10.

In one embodiment, the anti-BCMA antigen binding protein is an antibody comprising a HC comprising an amino acid sequence with at least 90%, 91%, 92%, 93%, 94%, 95%, 96%, 97%, 98%, 99% or 100% sequence identity to the amino acid sequence set forth in SEQ ID NO:9; and a LC comprising an amino acid sequence with at least 90%, 91%, 92%, 93%, 94%, 95%, 96%, 97%, 98%, 99% or 100% sequence identity to the amino acid sequence set forth in SEQ ID NO:10.

In one embodiment, the anti-BCMA antigen binding protein is an antibody comprising a HC comprising an amino acid sequence set forth in SEQ ID NO:9; and a LC comprising an amino acid sequence set forth in SEQ ID NO:10.

In one embodiment, the anti-BCMA antigen binding protein is an immunoconjugate comprising an antigen binding protein according to the invention as herein described including, but not limited to, an antibody conjugated to one or more cytotoxic agents, such as a chemotherapeutic agent, a drug, a growth inhibitory agent, a toxin (e.g., a protein toxin, an enzymatically active toxin of bacterial, fungal, plant, or animal origin, or fragments thereof), or a radioactive isotope (i.e., a radioconjugate). In a further embodiment the anti-BCMA antigen binding protein is conjugated to a toxin such as an auristatin, e.g., monomethyl auristatin E (MMAE) or monomethyl auristatin F (MMAF).

In one embodiment, the anti-BCMA antigen binding protein is an immunoconjugate having the following general structure:
ABP-((Linker)ₙ-Ctx)ₘ
wherein
   ABP is an antigen binding protein
   Linker is either absent or any a cleavable or non-cleavable linker
   Ctx is any cytotoxic agent described herein
   n is 0, 1, 2, or 3 and
   m is 1, 2, 3, 4, 5, 6, 7, 8, 9 or 10.

Exemplary linkers include 6-maleimidocaproyl (MC), maleimidopropanoyl (MP), valine-citrulline (val-cit), alanine-phenylalanine (ala-phe), p-aminobenzyloxycarbonyl (PAB), N-Succinimidyl 4-(2-pyridylthio)pentanoate (SPP), N-succinimidyl 4-(N-maleimidomethyl)cyclohexane-1 carboxylate (SMCC), and N-Succinimidyl (4-iodo-acetyl) aminobenzoate (SIAB).

In one embodiment, the anti-BCMA antigen binding protein is an immunoconjugate containing a monoclonal antibody linked to MMAE or MMAF. In another embodiment, the anti-BCMA antigen binding protein is an immunoconjugate containing a monoclonal antibody linked to MMAE or MMAF by an MC linker as depicted in the following structures:

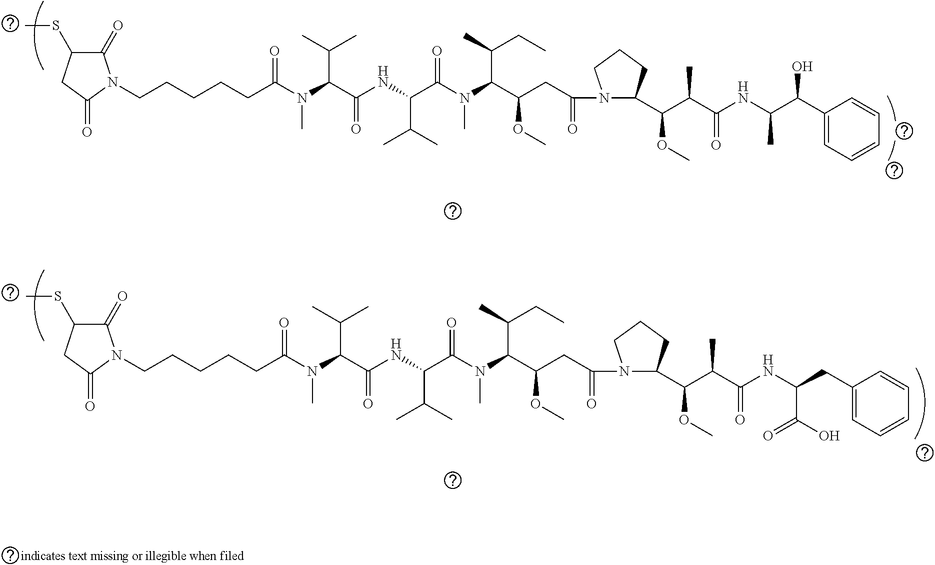 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)CCCCCN1C(=O)CC(SC)C1=O)C(C)C ; CC[C@H](C)[C@@H]([C@@H](CC(=O)N1CCC[C@H]1[C@H](OC)[C@@H](C)C(=O)N[C@@H](Cc1ccccc1)C(=O)O)OC)N(C)C(=O)[C@@H](NC(=O)[C@H](C(C)C)N(C)C(=O)CCCCCN1C(=O)CC(SC)C1=O)C(C)C]

In one aspect of the invention the anti-BCMA antigen binding protein is a chimeric antigen receptor. In a further aspect the CAR comprises a binding domain, a transmembrane domain and an intracellular effector domain.

In one aspect of the invention the anti-BCMA antigen binding protein is a bi-specific T-cell engager (BiTE) comprising a fusion protein consisting of two single-chain variable fragments (scFvs) of different antibodies.

The appropriate therapeutically effective dose of the anti-BCMA antigen binding protein will be determined readily by those of skill in the art. Suitable doses of the anti-BCMA antigen binding proteins described herein may be calculated for patients according to their weight, for example suitable doses may be in the range of about 0.1 mg/kg to about 20 mg/kg, for example about 1 mg/kg to about 20 mg/kg, for example about 10 mg/kg to about 20 mg/kg or for example about 1 mg/kg to about 15 mg/kg, for example about 10 mg/kg to about 15 mg/kg.

In one embodiment, the therapeutically effective dose of the anti-BCMA antigen binding protein is in the range of about 0.03 mg/kg to about 4.6 mg/kg. In yet another embodiment, the therapeutically effective dose of the anti-BCMA antigen binding protein is 0.03 mg/kg, 0.06 mg/kg, 0.12 mg/kg, 0.24 mg/kg, 0.48 mg/kg, 0.96 mg/kg, 1.92 mg/kg, 3.4 mg/kg, or 4.6 mg/kg. In yet another embodiment, the therapeutically effective dose of the anti-BCMA antigen binding protein is 1.9 mg/kg, 2.5 mg/kg or 3.4 mg/kg.

### Immunomodulatory Agents

The term “immunomodulatory agent” as used herein refers to an agent that induces, enhances, or suppresses an immune response. Immunomodulatory agents can be designed to elicit or amplify an immune response (activation immunomodulatory agents), or designed to reduce or suppress an immune response (suppression immunomodulatory agents). Examples of immunomodulatory agents include, but are not limited to, agents directed to ICOS and anti-CD38 antigen binding proteins.

### LCOS

In one embodiment, the immunomodulatory agent is agent directed to ICOS. As used herein “ICOS” means any Inducible T-cell costimulator protein. Pseudonyms for ICOS (Inducible T-cell COStimulator) include AILIM; CD278; CVID1, JTT-1 or JTT-2, MGC39850, or 8F4. ICOS is a CD28-superfamily costimulatory molecule that is expressed on activated T cells. The protein encoded by this gene belongs to the CD28 and CTLA-4 cell-surface receptor family. It forms homodimers and plays an important role in cell-cell signaling, immune responses, and regulation of cell proliferation. The amino acid sequence of human ICOS (isoform 2) (Accession No.: UniProtKB—Q9Y6W8-2) is represented in SEQ ID NO:11. The amino acid sequence of human ICOS (isoform 1) (Accession No.: UniProtKB - Q9Y6W8-1) is represented in SEQ ID NO:12.

Activation of ICOS occurs through binding by ICOS-L (B7RP-1/B7-H2). Neither B7-1 nor B7-2 (ligands for CD28 and CTLA4) bind or activate ICOS. However, ICOS-L has been shown to bind weakly to both CD28 and CTLA-4 (Yao S et al., “B7-H2 is a costimulatory ligand for CD28 in human”, Immunity, 34(5); 729-40 (2011)). Expression of ICOS appears to be restricted to T cells. ICOS expression levels vary between different T cell subsets and on T cell activation status. ICOS expression has been shown on resting TH17, T follicular helper (TFH) and regulatory T (Treg) cells; however, unlike CD28; it is not highly expressed on naive TH1 and TH2 effector T cell populations (Paulos C M et al., “The inducible costimulator (ICOS) is critical for the development of human Th17 cells”, Sci Transl Med, 2(55); 55ra78 (2010)). ICOS expression is highly induced on CD4+ and CD8+ effector T cells following activation through TCR engagement (Wakamatsu E, et al., “Convergent and divergent effects of costimulatory molecules in conventional and regulatory CD4+ T cells”, Proc Natal Acad Sci USA, 110(3); 1023-8 (2013)). Co-stimulatory signalling through ICOS receptor only occurs in T cells receiving a concurrent TCR activation signal (Sharpe A H and Freeman G J. “The B7-CD28 Superfamily”, Nat. Rev Immunol, 2(2); 116-26 (2002)). In activated antigen specific T cells, ICOS regulates the production of both TH1 and TH2 cytokines including IFN-γ, TNF-α, IL-10, IL-4, IL-13 and others. ICOS also stimulates effector T cell proliferation, albeit to a lesser extent than CD28 (Sharpe A H and Freeman G J. “The B7-CD28 Superfamily”, Nat. Rev Immunol, 2(2); 116-26 (2002)). Antibodies to ICOS and methods of using in the treatment of disease are described, for instance, in WO 2012/131004, US20110243929, and US20160215059. US20160215059 is incorporated by reference herein. CDRs for murine antibodies to human ICOS having agonist activity are shown in PCT/EP2012/055735 (WO 2012/131004). Antibodies to ICOS are also disclosed in WO 2008/137915, WO 2010/056804, EP 1374902, EP1374901, and EP1125585. Agonist antibodies to ICOS or ICOS binding proteins are disclosed in WO2012/13004, WO2014/033327, WO2016/120789, US20160215059, and US20160304610. Exemplary antibodies in US2016/0304610 include 37A10S713. Sequences of 37A10S713 are reproduced below as SEQ ID NOS: 21-28.

By “agent directed to ICOS” is meant any chemical compound or biological molecule capable of binding to ICOS. In some embodiments, the agent directed to ICOS is an ICOS binding protein. In some other embodiments, the agent directed to ICOS is an ICOS agonist.

As used herein “ICOS-L” and “ICOS Ligand” are used interchangeably and refer to the membrane bound natural ligand of human ICOS. ICOS ligand is a protein that in humans is encoded by the ICOSLG gene. ICOSLG has also been designated as CD275 (cluster of differentiation 275). Pseudonyms for ICOS-L include B7RP-1 and B7-H2.

The term “ICOS binding protein” as used herein refers to antibodies and other protein constructs, such as domains, which are capable of binding to ICOS. In some instances, the ICOS is human ICOS. The term “ICOS binding protein” can be used interchangeably with “ICOS antigen binding protein.”

Thus, as is understood in the art, anti-ICOS antibodies and/or ICOS antigen binding proteins would be considered ICOS binding proteins

In one embodiment, the immunomodulatory agent is an anti-ICOS antigen binding protein. In another embodiment, the immunomodulatory agent is an anti-ICOS antibody. In yet another embodiment, the anti-ICOS antibody is an agonist antibody directed to ICOS.

In one embodiment, the immunomodulatory agent is an anti-ICOS antibody comprising a heavy chain variable region CDR1 (“CDRH1”) comprising an amino acid sequence with at least 90%, 91%, 92%, 93%, 94%, 95%, 96%, 97%, 98%, 99% or 100% sequence identity to the amino acid sequence set forth in SEQ ID NO:13. In one embodiment, the heavy chain variable region CDR1 (“CDRH1”) comprises an amino acid sequence with one amino acid variation (variant) to the amino acid sequence set forth in SEQ ID NO:13.

In one embodiment, the immunomodulatory agent is an anti-ICOS antibody comprising a heavy chain variable region CDR2 (“CDRH2”) comprising an amino acid sequence with at least 90%, 91%, 92%, 93%, 94%, 95%, 96%, 97%, 98%, 99% or 100% sequence identity to the amino acid sequence set forth in SEQ ID NO:14. In one embodiment, the heavy chain variable region CDR2 (“CDRH2”) comprises an amino acid sequence with one amino acid variation (variant) to the amino acid sequence set forth in SEQ ID NO:14.

In one embodiment, the immunomodulatory agent is an anti-ICOS antibody comprising a heavy chain variable region CDR3 (“CDRH3”) comprising an amino acid sequence with at least 90%, 91%, 92%, 93%, 94%, 95%, 96%, 97%, 98%, 99% or 100% sequence identity to the amino acid sequence set forth in SEQ ID NO:15. In one embodiment, the heavy chain variable region CDR3 (“CDRH3”) comprises an amino acid sequence with one amino acid variation (variant) to the amino acid sequence set forth in SEQ ID NO:15.

In one embodiment, the immunomodulatory agent is an anti-ICOS antibody comprising a light chain variable region CDR1 (“CDRL1”) comprising an amino acid sequence with at least 90%, 91%, 92%, 93%, 94%, 95%, 96%, 97%, 98%, 99% or 100% sequence identity to the amino acid sequence set forth in SEQ ID NO:16. In one embodiment, the light chain variable region CDL1 (“CDR1”) comprises an amino acid sequence with one amino acid variation (variant) to the amino acid sequence set forth in SEQ ID NO:16.

In one embodiment, the immunomodulatory agent is an anti-ICOS antibody comprising a light chain variable region CDR2 (“CDRL2”) comprising an amino acid sequence with at least 90%, 91%, 92%, 93%, 94%, 95%, 96%, 97%, 98%, 99% or 100% sequence identity to the amino acid sequence set forth in SEQ ID NO:17. In one embodiment, the light chain variable region CDL2 (“CDR2”) comprises an amino acid sequence with one amino acid variation (variant) to the amino acid sequence set forth in SEQ ID NO:17.

In one embodiment, the immunomodulatory agent is an anti-ICOS antibody comprising a light chain variable region CDR3 (“CDRL3”) comprising an amino acid sequence with at least 90%, 91%, 92%, 93%, 94%, 95%, 96%, 97%, 98%, 99% or 100% sequence identity to the amino acid sequence set forth in SEQ ID NO:18. In one embodiment, the light chain variable region CDL3 (“CDR3”) comprises an amino acid sequence with one amino acid variation (variant) to the amino acid sequence set forth in SEQ ID NO:18.

In one embodiment, the immunomodulatory agent is an anti-ICOS antibody comprising a CDRH1 comprising an amino acid sequence with at least 90%, 91%, 92%, 93%, 94%, 95%, 96%, 97%, 98%, 99% or 100% sequence identity to the amino acid sequence set forth in SEQ ID NO:13; CDRH2 comprising an amino acid sequence with at least 90%, 91%, 92%, 93%, 94%, 95%, 96%, 97%, 98%, 99% or 100% sequence identity to the amino acid sequence set forth in SEQ ID NO:14; CDRH3 comprising an amino acid sequence with at least 90%, 91%, 92%, 93%, 94%, 95%, 96%, 97%, 98%, 99% or 100% sequence identity to the amino acid sequence set forth in SEQ ID NO:15; CDRL1 comprising an amino acid sequence with at least 90%, 91%, 92%, 93%, 94%, 95%, 96%, 97%, 98%, 99% or 100% sequence identity to the amino acid sequence set forth in SEQ ID NO:16; CDRL2 comprising an amino acid sequence with at least 90%, 91%, 92%, 93%, 94%, 95%, 96%, 97%, 98%, 99% or 100% sequence identity to the amino acid sequence set forth in SEQ ID NO:17; and/or CDRL3 comprising an amino acid sequence with at least 90%, 91%, 92%, 93%, 94%, 95%, 96%, 97%, 98%, 99% or 100% sequence identity to the amino acid sequence set forth in SEQ ID NO:18.

In one embodiment, the immunomodulatory agent is an anti-ICOS antibody comprising a CDRH1 comprising an amino acid sequence set forth in SEQ ID NO:13; a CDRH2 comprising an amino acid sequence set forth in SEQ ID NO:14; a CDRH3 comprising an amino acid sequence set forth in SEQ ID NO:15; a CDRL1 comprising an amino acid sequence set forth in SEQ ID NO:16; a CDRL2 comprising an amino acid sequence set forth in SEQ ID NO:17; and a CDRL3 comprising an amino acid sequence set forth in SEQ ID NO:18.

It will be appreciated that each of CDR H1, H2, H3, L1, L2, L3 of an anti-ICOS antibody may be modified alone or in combination with any other CDR, in any permutation or combination. In one embodiment, a CDR is modified by the substitution, deletion or addition of up to 3 amino acids, for example 1 or 2 amino acids, for example 1 amino acid. Typically, the modification is a substitution, particularly a conservative substitution, for example as shown in Table 1 below.

TABLE 1

| Side chain | Members |
|---|---|
| Hydrophobic | Met, Ala, Val, Leu, Ile |
| Neutral hydrophilic | Cys, Ser, Thr |
| Acidic | Asp, Glu |
| Basic | Asn, Gln, His, Lys, Arg |
| Residues that influence | Gly, Pro |
| chain orientation | |
| Aromatic | Trp, Tyr, Phe |

The subclass of an antibody in part determines secondary effector functions, such as complement activation or Fc receptor (FcR) binding and antibody dependent cell cytotoxicity (ADCC) (Huber, et al., Nature 229(5284): 419-20 (1971); Brunhouse, et al., Mol Immunol 16(11): 907-17 (1979)). In identifying the optimal type of antibody for a particular application, the effector functions of the antibodies can be taken into account. For example, hIgG1 antibodies have a relatively long half life, are very effective at fixing complement, and they bind to both FcγRT and FcγRII. In contrast, human IgG4 antibodies have a shorter half life, do not fix complement and have a lower affinity for the FcRs. Replacement of serine 228 with a proline (S228P) in the Fc region of IgG4 reduces heterogeneity observed with hIgG4 and extends the serum half life (Kabat, et al., “Sequences of proteins of immunological interest” 5.sup.th Edition (1991); Angal, et al., Mol Immunol 30(1): 105-8 (1993)). A second mutation that replaces leucine 235 with a glutamic acid (L235E) eliminates the residual FcR binding and complement binding activities (Alegre, et al., J Immunol 148(11): 3461-8 (1992)). The resulting antibody with both mutations is referred to as IgG4PE. The numbering of the hIgG4 amino acids was derived from EU numbering reference: Edelman, G. M. et al., Proc. Natl. Acad. USA, 63, 78-85 (1969). PMID: 5257969. In one embodiment of the present invention the anti-ICOS antibody is an IgG4 isotype. In one embodiment, the anti-ICOS antibody comprises an IgG4 Fc region comprising the replacement S228P and L235E may have the designation IgG4PE.

In one embodiment, the immunomodulatory agent is an anti-ICOS antibody comprising a heavy chain variable region (“VH”) comprising an amino acid sequence with at least 90%, 91%, 92%, 93%, 94%, 95%, 96%, 97%, 98%, 99% or 100% sequence identity to the amino acid sequence set forth in SEQ ID NO:19 and may be designated as “H2”.

In one embodiment, the immunomodulatory agent is an anti-ICOS antibody comprising a light chain variable region (“VL”) comprising an amino acid sequence with at least 90%, 91%, 92%, 93%, 94%, 95%, 96%, 97%, 98%, 99% or 100% sequence identity to the amino acid sequence set forth in SEQ ID NO:20 and may designated as “L5.”

In one embodiment, the immunomodulatory agent is an anti-ICOS antibody comprising a VH comprising an amino acid sequence with at least 90%, 91%, 92%, 93%, 94%, 95%, 96%, 97%, 98%, 99% or 100% sequence identity to the amino acid sequence set forth in SEQ ID NO:19; and a VL comprising an amino acid sequence with at least 90%, 91%, 92%, 93%, 94%, 95%, 96%, 97%, 98%, 99% or 100% sequence identity to the amino acid sequence set forth in SEQ ID NO:20 and may designated as “H2L5.”

In one embodiment, the immunomodulatory agent is an anti-ICOS antibody comprising a VH comprising an amino acid sequence set forth in SEQ ID NO:19; and a VL comprising an amino acid sequence set forth in SEQ ID NO:20 and may designated as “H2L5”.

In one aspect of the invention the agent directed to ICOS is a chimeric antigen receptor. In a further aspect, the CAR comprises a binding domain, a transmembrane domain and an intracellular effector domain.

In one aspect of the invention the agent directed to ICOS is a bi-specific T-cell engager (BiTE) comprising a fusion protein consisting of two single-chain variable fragments (scFvs) of different antibodies.

The appropriate therapeutically effective dose of the agent directed to ICOS will be determined readily by those of skill in the art. In one embodiment, the therapeutically effective dose of the agent directed to ICOS is in the range of about 0.0005 mg/kg to about 6 mg/kg. In another embodiment, therapeutically effective dose of the agent directed to

ICOS is in the range of about 0.001 mg/kg to about 3.0 mg/kg. In another embodiment, the therapeutically effective dose of the agent directed to ICOS is about 0.001 mg/kg, about 0.003 mg/kg, about 0.01 mg/kg, about 0.03 mg/kg, about 0.1 mg/kg, about 0.3 mg/kg, about 1.0 mg/kg, or about 3.0 mg/kg.

The appropriate therapeutically effective dose of the agent directed to ICOS will be determined readily by those of skill in the art. In one embodiment, the therapeutically effective dose of the agent directed to ICOS is in the range of about 0.04 mg to about 480 mg. In another embodiment, therapeutically effective dose of the agent directed to ICOS is in the range of about 0.08 mg to about 240 mg. In another embodiment, the therapeutically effective dose of the agent directed to ICOS is about 0.08 mg, about 0.24 mg, about 0.8 mg, about 2.4 mg, about 8 mg, about 24 mg, about 80 mg, or about 240 mg.

In one embodiment, the therapeutically effective dose of the agent directed to ICOS is about 80 mg. In another embodiment, the therapeutically effective dose of the agent directed to ICOS is about 24 mg. In still another embodiment, the therapeutically effective does of the agent directed to ICOS is about 240 mg. In another embodiment, the therapeutically effective does of the agent directed to ICOS is in the range of about 24 mg to about 240 mg.

### Anti-CD38 Antigen Binding Protein

In one embodiment, the immunomodulatory agent is anti-CD38 antigen binding protein. The anti-CD38 antigen binding proteins in the combinations described herein are useful in the treatment or prevention of cancers. The anti-CD38 antigen binding proteins described herein may bind to human CD38, for example, human CD38 containing the amino acid sequence of GenBank Accession Number D84284.2, or genes encoding human CD38 having at least 90 percent homology or at least 90 percent identity thereto.

CD38 is a transmembrane glycoprotein (48 kDa) expressed on the surface of hematopoietic cells, including multiple myeloma and other cell types and tissues and has multiple functions, such as receptor mediated adhesion, signaling, and modulation of cyclase and hydrolase activity. Without being bound by theory, it is believed that anti-CD38 antigen binding proteins, such as anti-CD38 antibodies, bind to CD38 and inhibit the growth of CD38 expressing tumor cells by inducing apoptosis directly through Fc mediated cross linking as well as by immune-mediated tumor cell lysis through complement dependent cytotoxicity (CDC), antibody dependent cell mediated cytotoxicity (ADCC) and antibody dependent cellular phagocytosis (ADCP).

The anti-CD38 antigen binding protein described herein includes antibodies, antibody fragments and other protein constructs which are capable of binding to CD38. The anti-CD38 antigen binding proteins of the present invention may comprise heavy chain variable regions and light chain variable regions of the invention which may be formatted into the structure of a natural antibody or functional fragment or equivalent thereof.

In one embodiment, the anti-CD38 antigen binding protein is an antibody. In another embodiment, the anto-CD38 antigen binding protein mediates killing of a CD38+target cell by antibody dependent cellular cytotoxicity. In yet another embodiment, the anti-CD38 antigen binding protein is an immunoglobulin G1 kappa (IgG1κ) human monoclonal antibody against CD38 antigen.

In one embodiment, the anti-CD38 antibody is daratumumab (Darzalex®—Janssen Biotech, Inc.)

Exemplary anti-CD38 antigen binding proteins and methods of making the same are disclosed in U.S. Pat. Nos. 8,263,746; 9,200,061; 8,088,896; 8,486,394; and 9,193,799, each of which is incorporated by reference herein in its entirety.

In one aspect of the invention the anti-CD38 antigen binding protein is a chimeric antigen receptor. In a further aspect, the CAR comprises a binding domain, a transmembrane domain and an intracellular effector domain.

In one aspect of the invention the anti-CD38 antigen binding protein is a bi-specific T-cell engager (BiTE) comprising a fusion protein consisting of two single-chain variable fragments (scFvs) of different antibodies.

The appropriate therapeutically effective dose of the anti-CD38 antigen binding protein will be determined readily by those of skill in the art. Suitable doses of the anti-CD38 antigen binding proteins described herein may be calculated for patients according to their weight, for example suitable doses may be in the range of about 0.1 mg/kg to about 30 mg/kg, for example about 5 mg/kg to about 20 mg/kg, or for example about 10 mg/kg to about 20 mg/kg.

In one embodiment, the therapeutically effective dose of the anti-CD38 antigen binding protein is about 5 mg/kg, about 6 mg/kg, about 7 mg/kg, about 8 mg/kg, about 9 mg/kg, about 10 mg/kg, about 11 mg/kg, about 12 mg/kg, about 13 mg/kg, about 14 mg/kg, about 15 mg/kg, about 16 mg/kg, about 17 mg/kg, about 18 mg/kg, about 19 mg/kg, or about 20 mg/kg. In yet another embodiment, the therapeutically effective dose of the anti-CD38 antigen binding protein is about 16 mg/kg.

### Methods of Treatment

Described herein are methods for treating cancer in a subject with the combinations described herein. As used herein, the terms “cancer,” and “tumor” are used interchangeably and, in either the singular or plural form, refer to cells that have undergone a malignant transformation that makes them pathological to the host organism. Primary cancer cells can be readily distinguished from non-cancerous cells by well-established techniques, particularly histological examination. The definition of a cancer cell, as used herein, includes not only a primary cancer cell, but any cell derived from a cancer cell ancestor. This includes metastasized cancer cells, and in vitro cultures and cell lines derived from cancer cells. When referring to a type of cancer that normally manifests as a solid tumor, a “clinically detectable” tumor is one that is detectable on the basis of tumor mass; e.g., by procedures such as computed tomography (CT) scan, magnetic resonance imaging (MRI), X-ray, ultrasound or palpation on physical examination, and/or which is detectable because of the expression of one or more cancer-specific antigens in a sample obtainable from a patient. Tumors may be a hematopoietic (or hematologic or hematological or blood-related) cancer, for example, cancers derived from blood cells or immune cells, which may be referred to as “liquid tumors.” Specific examples of clinical conditions based on hematologic tumors include leukemias such as chronic myelocytic leukemia, acute myelocytic leukemia, chronic lymphocytic leukemia and acute lymphocytic leukemia; plasma cell malignancies such as multiple myeloma, MGUS and Waldenstrom's macroglobulinemia; lymphomas such as non-Hodgkin's lymphoma, Hodgkin's lymphoma; and the like.

The cancer may be any in which an abnormal number of blast cells or unwanted cell proliferation is present or that is diagnosed as a hematological cancer, including both lymphoid and myeloid malignancies. Myeloid malignancies include, but are not limited to, acute myeloid (or myelocytic or myelogenous or myeloblastic) leukemia (undifferentiated or differentiated), acute promyeloid (or promyelocytic or promyelogenous or promyeloblastic) leukemia, acute myelomonocytic (or myelomonoblastic) leukemia, acute monocytic (or monoblastic) leukemia, erythroleukemia and megakaryocytic (or megakaryoblastic) leukemia. These leukemias may be referred together as acute myeloid (or myelocytic or myelogenous) leukemia (AML). Myeloid malignancies also include myeloproliferative disorders (MPD) which include, but are not limited to, chronic myelogenous (or myeloid) leukemia (CML), chronic myelomonocytic leukemia (CMML), essential thrombocythemia (or thrombocytosis), and polcythemia vera (PCV). Myeloid malignancies also include myelodysplasia (or myelodysplastic syndrome or MDS), which may be referred to as refractory anemia (RA), refractory anemia with excess blasts (RAEB), and refractory anemia with excess blasts in transformation (RAEBT); as well as myelofibrosis (MFS) with or without agnogenic myeloid metaplasia.

Hematopoietic cancers also include lymphoid malignancies, which may affect the lymph nodes, spleens, bone marrow, peripheral blood, and/or extranodal sites. Lymphoid cancers include B-cell malignancies, which include, but are not limited to, B-cell non-Hodgkin's lymphomas (B-NHLs). B-NHLs may be indolent (or low-grade), intermediate-grade (or aggressive) or high-grade (very aggressive). Indolent B-cell lymphomas include follicular lymphoma (FL); small lymphocytic lymphoma (SLL); marginal zone lymphoma (MZL) including nodal MZL, extranodal MZL, splenic MZL and splenic MZL with villous lymphocytes; lymphoplasmacytic lymphoma (LPL); and mucosa-associated-lymphoid tissue (MALT or extranodal marginal zone) lymphoma. Intermediate-grade B-NHLs include mantle cell lymphoma (MCL) with or without leukemic involvement, diffuse large cell lymphoma (DLBCL), follicular large cell (or grade 3 or grade 3B) lymphoma, and primary mediastinal lymphoma (PML). High-grade B-NHLs include Burkitt's lymphoma (BL), Burkitt-like lymphoma, small non-cleaved cell lymphoma (SNCCL) and lymphoblastic lymphoma. Other B-NHLs include immunoblastic lymphoma (or immunocytoma), primary effusion lymphoma, HIV associated (or AIDS related) lymphomas, and post-transplant lymphoproliferative disorder (PTLD) or lymphoma. B-cell malignancies also include, but are not limited to, chronic lymphocytic leukemia (CLL), prolymphocytic leukemia (PLL), Waldenstrom's macroglobulinemia (WM), hairy cell leukemia (HCL), large granular lymphocyte (LGL) leukemia, acute lymphoid (or lymphocytic or lymphoblastic) leukemia, and Castleman's disease. NHL may also include T-cell non-Hodgkin's lymphoma s(T-NHLs), which include, but are not limited to T-cell non-Hodgkin's lymphoma not otherwise specified (NOS), peripheral T-cell lymphoma (PTCL), anaplastic large cell lymphoma (ALCL), angioimmunoblastic lymphoid disorder (AILD), nasal natural killer (NK) cell/T-cell lymphoma, gamma/delta lymphoma, cutaneous T cell lymphoma, mycosis fungoides, and Sezary syndrome.

Hematopoietic cancers also include Hodgkin's lymphoma (or disease) including classical Hodgkin's lymphoma, nodular sclerosing Hodgkin's lymphoma, mixed cellularity Hodgkin's lymphoma, lymphocyte predominant (LP) Hodgkin's lymphoma, nodular LP Hodgkin's lymphoma, and lymphocyte depleted Hodgkin's lymphoma. Hematopoietic cancers also include plasma cell diseases or cancers such as multiple myeloma (MM) including smoldering MM, monoclonal gammopathy of undetermined (or unknown or unclear) significance (MGUS), plasmacytoma (bone, extramedullary), lymphoplasmacytic lymphoma (LPL), Waldenstroem's Macroglobulinemia, plasma cell leukemia, and primary amyloidosis (AL). Hematopoietic cancers may also include other cancers of additional hematopoietic cells, including polymorphonuclear leukocytes (or neutrophils), basophils, eosinophils, dendritic cells, platelets, erythrocytes and natural killer cells. Tissues which include hematopoietic cells referred herein to as “hematopoietic cell tissues” include bone marrow; peripheral blood; thymus; and peripheral lymphoid tissues, such as spleen, lymph nodes, lymphoid tissues associated with mucosa (such as the gut-associated lymphoid tissues), tonsils, Peyer's patches and appendix, and lymphoid tissues associated with other mucosa, for example, the bronchial linings.

In one embodiment, the cancer is selected from the group consisting of colorectal cancer (CRC), gastric, esophageal, cervical, bladder, breast, head and neck, ovarian, melanoma, renal cell carcinoma (RCC), EC squamous cell, non-small cell lung carcinoma, mesothelioma, pancreatic, and prostate cancer.

The term “treating” and derivatives thereof as used herein, is meant to include therapeutic therapy. In reference to a particular condition, treating means: (1) to ameliorate the condition or one or more of the biological manifestations of the condition; (2) to interfere with (a) one or more points in the biological cascade that leads to or is responsible for the condition or (b) one or more of the biological manifestations of the condition; (3) to alleviate one or more of the symptoms, effects or side effects associated with the condition or one or more of the symptoms, effects or side effects associated with the condition or treatment thereof; (4) to slow the progression of the condition or one or more of the biological manifestations of the condition and/or (5) to cure said condition or one or more of the biological manifestations of the condition by eliminating or reducing to undetectable levels one or more of the biological manifestations of the condition for a period of time considered to be a state of remission for that manifestation without additional treatment over the period of remission. One skilled in the art will understand the duration of time considered to be remission for a particular disease or condition.

Prophylactic therapy is also contemplated. The skilled artisan will appreciate that “prevention” is not an absolute term. In medicine, “prevention” is understood to refer to the prophylactic administration of a drug to substantially diminish the likelihood or severity of a condition or biological manifestation thereof, or to delay the onset of such condition or biological manifestation thereof Prophylactic therapy is appropriate, for example, when a subject is considered at high risk for developing cancer, such as when a subject has a strong family history of cancer or when a subject has been exposed to a carcinogen.

“Subject” is defined broadly to include any patient in need of treatment, for example, a patient in need of cancer treatment. A subject may include a mammal. In one embodiment, the subject is a human patient. The subject in need of cancer treatment may include patients from a variety of stages including newly diagnosed, relapsed, refractory, progressive disease, remission, and others. The subject in need of cancer treatment may also include patients who have undergone stem cell transplant or who are considered transplant ineligible.

Subjects may be pre-screened in order to be selected for treatment with the combinations described herein. In one embodiment, a sample from the subject is tested for expression of BCMA prior to treatment with the combinations described herein.

Subjects may have had at least one prior cancer therapy before being treated with the combinations of the present invention. In one embodiment, the subject has been treated with at least 1, at least 2, at least 3, at least 4, at least 5, at least 6, or at least 7 prior cancer therapies before being treated with the combinations of the present invention.

In another embodiment, the subject has newly diagnosed cancer and has had 0 prior therapies before being treated with the combinations of the present invention.

The individual therapeutic agents of the combination of the invention, and pharmaceutical compositions comprising such therapeutic agents may be administered together or separately. When administered separately, this may occur simultaneously or sequentially in any order (by the same or by different routes of administration). Such sequential administration may be close in time or remote in time. The dose of a therapeutic agents of the invention or pharmaceutically acceptable salt thereof and the further therapeutically active agent(s) and the relative timings of administration will be selected in order to achieve the desired combined therapeutic effect.

The therapeutic agents of the invention may be administered by any appropriate route. For some therapeutic agents, suitable routes include oral, rectal, nasal, topical (including buccal and sublingual), vaginal, and parenteral (including subcutaneous, intramuscular, intraveneous, intradermal, intrathecal, and epidural). It will be appreciated that the preferred route may vary with, for example, the condition of the recipient of the combination and the cancer to be treated. It will also be appreciated that each of the agents administered may be administered by the same or different routes and that the therapeutic agents may be formulated together or in separate pharmaceutical compositions.

In one embodiment, one or more therapeutic agents of a combination of the invention are administered intravenously. In another embodiment, one or more therapeutic agents of a combination of the invention are administered intratumorally. In another embodiment, one or more therapeutic agents of a combination of the invention are administered orally. In another embodiment, one or more therapeutic agents of a combination of the invention are administered systemically, e.g., intravenously, and one or more other therapeutic agents of a combination of the invention are administered intratumorally. In another embodiment, all of the therapeutic agents of a combination of the invention are administered systemically, e.g., intravenously. In an alternative embodiment, all of the therapeutic agents of the combination of the invention are administered intratumorally. In any of the embodiments, e.g., in this paragraph, the therapeutic agents of the invention are administered as one or more pharmaceutical compositions.

In one embodiment, the invention provides a method of treating cancer in a subject in need thereof by administering a therapeutically effective dose of a combination described herein.

In one embodiment, the invention provides a method of treating cancer in a subject in need thereof by administering a therapeutically effective dose of a combination comprising an anti-BCMA antigen binding protein and an immunomodulatory agent.

In one embodiment, the invention provides a method of treating cancer in a subject in need thereof by administering a therapeutically effective dose of a combination comprising an anti-BCMA antigen binding protein and an agent directed to ICOS. In another embodiment, the invention provides a method of treating cancer in a subject in need thereof by administering a therapeutically effective dose of a combination comprising an anti-BCMA antibody and an anti-ICOS antibody.

In one embodiment, the invention provides a method of treating cancer in a subject in need thereof by administering a therapeutically effective dose of a combination comprising an anti-BCMA antigen binding protein and an anit-CD38 antigen binding protein. In another embodiment, the invention provides a method of treating cancer in a subject in need thereof by administering a therapeutically effective dose of a combination comprising an anti-BCMA antibody and an anti-CD38 antibody. In yet another embodiment, the invention provides a method of treating cancer in a subject in need thereof by administering a therapeutically effective dose of a combination comprising an anti-BCMA antibody and daratumumab.

In one embodiment, the invention provides a method of treating cancer in a subject in need thereof by administering a therapeutically effective dose of a combination comprising an anti-BCMA antibody and an agent directed to ICOS, wherein the anti-BCMA antibody comprises a CDRH1 comprising an amino acid sequence with at least 90%, 91%, 92%, 93%, 94%, 95%, 96%, 97%, 98%, 99% or 100% sequence identity to the amino acid sequence set forth in SEQ ID NO:1; a CDRH2 comprising an amino acid sequence with at least 90%, 91%, 92%, 93%, 94%, 95%, 96%, 97%, 98%, 99% or 100% sequence identity to the amino acid sequence set forth in SEQ ID NO:2; a CDRH3 comprising an amino acid sequence with at least 90%, 91%, 92%, 93%, 94%, 95%, 96%, 97%, 98%, 99% or 100% sequence identity to the amino acid sequence set forth in SEQ ID NO:3; a CDRL1 comprising an amino acid sequence with at least 90%, 91%, 92%, 93%, 94%, 95%, 96%, 97%, 98%, 99% or 100% sequence identity to the amino acid sequence set forth in SEQ ID NO:4; a CDRL2 comprising an amino acid sequence with at least 90%, 91%, 92%, 93%, 94%, 95%, 96%, 97%, 98%, 99% or 100% sequence identity to the amino acid sequence set forth in SEQ ID NO:5; and/or a CDRL3 comprising an amino acid sequence with at least 90%, 91%, 92%, 93%, 94%, 95%, 96%, 97%, 98%, 99% or 100% sequence identity to the amino acid sequence set forth in SEQ ID NO:6.

In one embodiment, the invention provides a method of treating cancer in a subject in need thereof by administering a therapeutically effective dose of a combination comprising an anti-BCMA antibody and an agent directed to ICOS, wherein the anti-BCMA antibody comprises a VH comprising an amino acid sequence with at least 90%, 91%, 92%, 93%, 94%, 95%, 96%, 97%, 98%, 99% or 100% sequence identity to the amino acid sequence set forth in SEQ ID NO:7; and/or a VL comprising an amino acid sequence with at least 90%, 91%, 92%, 93%, 94%, 95%, 96%, 97%, 98%, 99% or 100% sequence identity to the amino acid sequence set forth in SEQ ID NO:8.

In one embodiment, the invention provides a method of treating cancer in a subject in need thereof by administering a therapeutically effective dose of a combination comprising an anti-BCMA antibody and an agent directed to ICOS, wherein the anti-BCMA antibody comprises a HC comprising an amino acid sequence with at least 90%, 91%, 92%, 93%, 94%, 95%, 96%, 97%, 98%, 99% or 100% sequence identity to the amino acid sequence set forth in SEQ ID NO:9; and/or a LC comprising an amino acid sequence with at least 90%, 91%, 92%, 93%, 94%, 95%, 96%, 97%, 98%, 99% or 100% sequence identity to the amino acid sequence set forth in SEQ ID NO:10.

In one embodiment, the invention provides a method of treating cancer in a subject in need thereof by administering a therapeutically effective dose of a combination comprising an anti-BCMA antibody and an anti-ICOS antibody, wherein the anti-ICOS antibody comprises a CDRH1 comprising an amino acid sequence with at least 90%, 91%, 92%, 93%, 94%, 95%, 96%, 97%, 98%, 99% or 100% sequence identity to the amino acid sequence set forth in SEQ ID NO:13; a CDRH2 comprising an amino acid sequence with at least 90%, 91%, 92%, 93%, 94%, 95%, 96%, 97%, 98%, 99% or 100% sequence identity to the amino acid sequence set forth in SEQ ID NO:14; a CDRH3 comprising an amino acid sequence with at least 90%, 91%, 92%, 93%, 94%, 95%, 96%, 97%, 98%, 99% or 100% sequence identity to the amino acid sequence set forth in SEQ ID NO:15; a CDRL1 comprising an amino acid sequence with at least 90%, 91%, 92%, 93%, 94%, 95%, 96%, 97%, 98%, 99% or 100% sequence identity to the amino acid sequence set forth in SEQ ID NO:16; a CDRL2 comprising an amino acid sequence with at least 90%, 91%, 92%, 93%, 94%, 95%, 96%, 97%, 98%, 99% or 100% sequence identity to the amino acid sequence set forth in SEQ ID NO:17; and/or a CDRL3 comprising an amino acid sequence with at least 90%, 91%, 92%, 93%, 94%, 95%, 96%, 97%, 98%, 99% or 100% sequence identity to the amino acid sequence set forth in SEQ ID NO:18.

In one embodiment, the invention provides a method of treating cancer in a subject in need thereof by administering a therapeutically effective dose of a combination comprising an anti-BCMA antibody and an anti-ICOS antibody, wherein the anti-ICOS antibody comprises a VH comprising an amino acid sequence with at least 90%, 91%, 92%, 93%, 94%, 95%, 96%, 97%, 98%, 99% or 100% sequence identity to the amino acid sequence set forth in SEQ ID NO:19; and/or a VL comprising an amino acid sequence with at least 90%, 91%, 92%, 93%, 94%, 95%, 96%, 97%, 98%, 99% or 100% sequence identity to the amino acid sequence set forth in SEQ ID NO:20.

In one embodiment, the invention provides a method of treating cancer in a subject in need thereof by administering a therapeutically effective dose of a combination comprising an anti-BCMA antibody and an anti-ICOS antibody, wherein the anti-BCMA antibody comprises a CDRH1 comprising an amino acid sequence set forth in SEQ ID NO:1; a CDRH2 comprising an amino acid sequence set forth in SEQ ID NO:2; a CDRH3 comprising an amino acid sequence set forth in SEQ ID NO:3; a CDRL1 comprising an amino acid sequence set forth in SEQ ID NO:4; a CDRL2 comprising an amino acid sequence set forth in SEQ ID NO:5; and/or a CDRL3 comprising an amino acid sequence set forth in SEQ ID NO:6; and wherein the anti-ICOS antibody comprises a CDRH1 comprising an amino acid sequence set forth in SEQ ID NO:13; a CDRH2 comprising an amino acid sequence set forth in SEQ ID NO:14; a CDRH3 comprising an amino acid sequence set forth in SEQ ID NO:15; a CDRL1 comprising an amino acid sequence set forth in SEQ ID NO:16; a CDRL2 comprising an amino acid sequence set forth in SEQ ID NO:17; and/or a CDRL3 comprising an amino acid sequence set forth in SEQ ID NO:18.

In one embodiment, the invention provides a method of treating cancer in a subject in need thereof by administering a therapeutically effective dose of a combination comprising an anti-BCMA antibody and an anti-ICOS antibody, wherein the anti-BCMA antibody comprises a VH comprising an amino acid sequence set forth in SEQ ID NO:7; and/or a VL comprising an amino acid sequence set forth in SEQ ID NO:8; and wherein the anti-ICOS antibody comprises a VH comprising an amino acid sequence set forth in SEQ ID NO:19; and/or a VL comprising an amino acid sequence set forth in SEQ ID NO:20.

In one embodiment, the invention provides a method of treating cancer in a subject in need thereof by administering a therapeutically effective dose of a combination comprising an anti-BCMA antibody and an anti-CD38 antibody, wherein the anti-BCMA antibody comprises a CDRH1 comprising an amino acid sequence with at least 90%, 91%, 92%, 93%, 94%, 95%, 96%, 97%, 98%, 99% or 100% sequence identity to the amino acid sequence set forth in SEQ ID NO:1; a CDRH2 comprising an amino acid sequence with at least 90%, 91%, 92%, 93%, 94%, 95%, 96%, 97%, 98%, 99% or 100% sequence identity to the amino acid sequence set forth in SEQ ID NO:2; a CDRH3 comprising an amino acid sequence with at least 90%, 91%, 92%, 93%, 94%, 95%, 96%, 97%, 98%, 99% or 100% sequence identity to the amino acid sequence set forth in SEQ ID NO:3; a CDRL1 comprising an amino acid sequence with at least 90%, 91%, 92%, 93%, 94%, 95%, 96%, 97%, 98%, 99% or 100% sequence identity to the amino acid sequence set forth in SEQ ID NO:4; a CDRL2 comprising an amino acid sequence with at least 90%, 91%, 92%, 93%, 94%, 95%, 96%, 97%, 98%, 99% or 100% sequence identity to the amino acid sequence set forth in SEQ ID NO:5; and/or a CDRL3 comprising an amino acid sequence with at least 90%, 91%, 92%, 93%, 94%, 95%, 96%, 97%, 98%, 99% or 100% sequence identity to the amino acid sequence set forth in SEQ ID NO:6.

In one embodiment, the invention provides a method of treating cancer in a subject in need thereof by administering a therapeutically effective dose of a combination comprising an anti-BCMA antibody and an anti-CD38 antibody, wherein the anti-BCMA antibody comprises a VH comprising an amino acid sequence with at least 90%, 91%, 92%, 93%, 94%, 95%, 96%, 97%, 98%, 99% or 100% sequence identity to the amino acid sequence set forth in SEQ ID NO:7; and/or a VL comprising an amino acid sequence with at least 90%, 91%, 92%, 93%, 94%, 95%, 96%, 97%, 98%, 99% or 100% sequence identity to the amino acid sequence set forth in SEQ ID NO:8.

In one embodiment, the invention provides a method of treating cancer in a subject in need thereof by administering a therapeutically effective dose of a combination comprising an anti-BCMA antibody and an anti-CD38 antibody, wherein the anti-BCMA antibody comprises a HC comprising an amino acid sequence with at least 90%, 91%, 92%, 93%, 94%, 95%, 96%, 97%, 98%, 99% or 100% sequence identity to the amino acid sequence set forth in SEQ ID NO:9; and/or a LC comprising an amino acid sequence with at least 90%, 91%, 92%, 93%, 94%, 95%, 96%, 97%, 98%, 99% or 100% sequence identity to the amino acid sequence set forth in SEQ ID NO:10.

In one embodiment, the invention provides a method of treating cancer in a subject in need thereof by administering a therapeutically effective dose of a combination comprising an anti-BCMA antibody and daratumumab, wherein the anti-BCMA antibody comprises a CDRH1 comprising an amino acid sequence set forth in SEQ ID NO:1; a CDRH2 comprising an amino acid sequence set forth in SEQ ID NO:2; a CDRH3 comprising an amino acid sequence set forth in SEQ ID NO:3; a CDRL1 comprising an amino acid sequence set forth in SEQ ID NO:4; a CDRL2 comprising an amino acid sequence set forth in SEQ ID NO:5; and/or a CDRL3 comprising an amino acid sequence set forth in SEQ ID NO:6.

In one embodiment, the invention provides a method of treating cancer in a subject in need thereof by administering a therapeutically effective dose of a combination comprising an anti-BCMA antibody and daratumumab, wherein the anti-BCMA antibody comprises a VH comprising an amino acid sequence set forth in SEQ ID NO:7; and/or a VL comprising an amino acid sequence set forth in SEQ ID NO:8.

In one embodiment, the invention provides a combination, as described herein, for use in therapy.

In one embodiment, the invention provides a combination, as described herein, for use in the treatment of cancer.

In one embodiment, the invention provides a combination, as described herein, for use in the treatment of cancer, wherein the combination comprises an anti-BCMA antigen binding protein and an immunomodulatory agent.

In one embodiment, the invention provides a combination, as described herein, for use in the treatment of cancer, wherein the combination comprises an anti-BCMA antibody and an agent directed to ICOS, wherein the anti-BCMA antibody comprises a CDRH1 comprising an amino acid sequence with at least 90%, 91%, 92%, 93%, 94%, 95%, 96%, 97%, 98%, 99% or 100% sequence identity to the amino acid sequence set forth in SEQ ID NO:1; a CDRH2 comprising an amino acid sequence with at least 90%, 91%, 92%, 93%, 94%, 95%, 96%, 97%, 98%, 99% or 100% sequence identity to the amino acid sequence set forth in SEQ ID NO:2; a CDRH3 comprising an amino acid sequence with at least 90%, 91%, 92%, 93%, 94%, 95%, 96%, 97%, 98%, 99% or 100% sequence identity to the amino acid sequence set forth in SEQ ID NO:3; a CDRL1 comprising an amino acid sequence with at least 90%, 91%, 92%, 93%, 94%, 95%, 96%, 97%, 98%, 99% or 100% sequence identity to the amino acid sequence set forth in SEQ ID NO:4; a CDRL2 comprising an amino acid sequence with at least 90%, 91%, 92%, 93%, 94%, 95%, 96%, 97%, 98%, 99% or 100% sequence identity to the amino acid sequence set forth in SEQ ID NO:5; and/or a CDRL3 comprising an amino acid sequence with at least 90%, 91%, 92%, 93%, 94%, 95%, 96%, 97%, 98%, 99% or 100% sequence identity to the amino acid sequence set forth in SEQ ID NO:6.

In one embodiment, the invention provides a combination, as described herein, for use in the treatment of cancer, wherein the combination comprises an anti-BCMA antibody and an agent directed to ICOS, wherein the anti-BCMA antibody comprises a VH comprising an amino acid sequence with at least 90%, 91%, 92%, 93%, 94%, 95%, 96%, 97%, 98%, 99% or 100% sequence identity to the amino acid sequence set forth in SEQ ID NO:7; and/or a VL comprising an amino acid sequence with at least 90%, 91%, 92%, 93%, 94%, 95%, 96%, 97%, 98%, 99% or 100% sequence identity to the amino acid sequence set forth in SEQ ID NO:8.

In one embodiment, the invention provides a combination, as described herein, for use in the treatment of cancer, wherein the combination comprises an anti-BCMA antibody and an agent directed to ICOS, wherein the anti-BCMA antibody has comprises a HC comprising an amino acid sequence with at least 90%, 91%, 92%, 93%, 94%, 95%, 96%, 97%, 98%, 99% or 100% sequence identity to the amino acid sequence set forth in SEQ ID NO:9; and/or a LC comprising an amino acid sequence with at least 90%, 91%, 92%, 93%, 94%, 95%, 96%, 97%, 98%, 99% or 100% sequence identity to the amino acid sequence set forth in SEQ ID NO:10.

In one embodiment, the invention provides a combination, as described herein, for use in the treatment of cancer, wherein the combination comprises an anti-BCMA antibody and an anti-ICOS antibody, wherein the anti-ICOS antibody comprises a CDRH1 comprising an amino acid sequence with at least 90%, 91%, 92%, 93%, 94%, 95%, 96%, 97%, 98%, 99% or 100% sequence identity to the amino acid sequence set forth in SEQ ID NO:13; a CDRH2 comprising an amino acid sequence with at least 90%, 91%, 92%, 93%, 94%, 95%, 96%, 97%, 98%, 99% or 100% sequence identity to the amino acid sequence set forth in SEQ ID NO:14; a CDRH3 comprising an amino acid sequence with at least 90%, 91%, 92%, 93%, 94%, 95%, 96%, 97%, 98%, 99% or 100% sequence identity to the amino acid sequence set forth in SEQ ID NO:15; a CDRL1 comprising an amino acid sequence with at least 90%, 91%, 92%, 93%, 94%, 95%, 96%, 97%, 98%, 99% or 100% sequence identity to the amino acid sequence set forth in SEQ ID NO:16; a CDRL2 comprising an amino acid sequence with at least 90%, 91%, 92%, 93%, 94%, 95%, 96%, 97%, 98%, 99% or 100% sequence identity to the amino acid sequence set forth in SEQ ID NO:17; and/or a CDRL3 comprising an amino acid sequence with at least 90%, 91%, 92%, 93%, 94%, 95%, 96%, 97%, 98%, 99% or 100% sequence identity to the amino acid sequence set forth in SEQ ID NO:18.

In one embodiment, the invention provides a combination, as described herein, for use in the treatment of cancer, wherein the combination comprises an anti-BCMA antibody and an anti-ICOS antibody, wherein the anti-ICOS antibody comprises a VH comprising an amino acid sequence with at least 90%, 91%, 92%, 93%, 94%, 95%, 96%, 97%, 98%, 99% or 100% sequence identity to the amino acid sequence set forth in SEQ ID NO:19; and/or a VL comprising an amino acid sequence with at least 90%, 91%, 92%, 93%, 94%, 95%, 96%, 97%, 98%, 99% or 100% sequence identity to the amino acid sequence set forth in SEQ ID NO:20.

In one embodiment, the invention provides a combination, as described herein, for use in the treatment of cancer, wherein the combination comprises an anti-BCMA antibody and an anti-ICOS antibody, wherein the anti-BCMA antibody comprises a CDRH1 comprising an amino acid sequence set forth in SEQ ID NO:1; a CDRH2 comprising an amino acid sequence set forth in SEQ ID NO:2; a CDRH3 comprising an amino acid sequence set forth in SEQ ID NO:3; a CDRL1 comprising an amino acid sequence set forth in SEQ ID NO:4; a CDRL2 comprising an amino acid sequence set forth in SEQ ID NO:5; and/or a CDRL3 comprising an amino acid sequence set forth in SEQ ID NO:6; wherein the anti-ICOS antibody comprises a CDRH1 comprising an amino acid sequence set forth in SEQ ID NO:13; a CDRH2 comprising an amino acid sequence set forth in SEQ ID NO:14; a CDRH3 comprising an amino acid sequence set forth in SEQ ID NO:15; a CDRL1 comprising an amino acid sequence set forth in SEQ ID NO:16; a CDRL2 comprising an amino acid sequence set forth in SEQ ID NO:17; and/or a CDRL3 comprising an amino acid sequence set forth in SEQ ID NO:18.

In one embodiment, the invention provides a combination, as described herein, for use in the treatment of cancer, wherein the combination comprises an anti-BCMA antibody and an anti-ICOS antibody, wherein the anti-BCMA antibody comprises a VH comprising an amino acid sequence set forth in SEQ ID NO:7; and/or a VL comprising an amino acid sequence set forth in SEQ ID NO:8; and wherein the anti-ICOS antibody comprises a VH comprising an amino acid sequence set forth in SEQ ID NO:19; and/or a VL comprising an amino acid sequence set forth in SEQ ID NO:20.

In one embodiment, the invention provides a combination, as described herein, for use in the treatment of cancer, wherein the combination comprises an anti-BCMA antibody and an anti-CD38 antibody, wherein the anti-BCMA antibody comprises a CDRH1 comprising an amino acid sequence with at least 90%, 91%, 92%, 93%, 94%, 95%, 96%, 97%, 98%, 99% or 100% sequence identity to the amino acid sequence set forth in SEQ ID NO:1; a CDRH2 comprising an amino acid sequence with at least 90%, 91%, 92%, 93%, 94%, 95%, 96%, 97%, 98%, 99% or 100% sequence identity to the amino acid sequence set forth in SEQ ID NO:2; a CDRH3 comprising an amino acid sequence with at least 90%, 91%, 92%, 93%, 94%, 95%, 96%, 97%, 98%, 99% or 100% sequence identity to the amino acid sequence set forth in SEQ ID NO:3; a CDRL1 comprising an amino acid sequence with at least 90%, 91%, 92%, 93%, 94%, 95%, 96%, 97%, 98%, 99% or 100% sequence identity to the amino acid sequence set forth in SEQ ID NO:4; a CDRL2 comprising an amino acid sequence with at least 90%, 91%, 92%, 93%, 94%, 95%, 96%, 97%, 98%, 99% or 100% sequence identity to the amino acid sequence set forth in SEQ ID NO:5; and/or a CDRL3 comprising an amino acid sequence with at least 90%, 91%, 92%, 93%, 94%, 95%, 96%, 97%, 98%, 99% or 100% sequence identity to the amino acid sequence set forth in SEQ ID NO:6.

In one embodiment, the invention provides a combination, as described herein, for use in the treatment of cancer, wherein the combination comprises an anti-BCMA antibody and an anti-CD38 antibody, wherein the anti-BCMA antibody comprises a VH comprising an amino acid sequence with at least 90%, 91%, 92%, 93%, 94%, 95%, 96%, 97%, 98%, 99% or 100% sequence identity to the amino acid sequence set forth in SEQ ID NO:7; and/or a VL comprising an amino acid sequence with at least 90%, 91%, 92%, 93%, 94%, 95%, 96%, 97%, 98%, 99% or 100% sequence identity to the amino acid sequence set forth in SEQ ID NO:8.

In one embodiment, the invention provides a combination, as described herein, for use in the treatment of cancer, wherein the combination comprises an anti-BCMA antibody and an anti-CD38 antibody, wherein the anti-BCMA antibody comprises a HC comprising an amino acid sequence with at least 90%, 91%, 92%, 93%, 94%, 95%, 96%, 97%, 98%, 99% or 100% sequence identity to the amino acid sequence set forth in SEQ ID NO:9; and/or a LC comprising an amino acid sequence with at least 90%, 91%, 92%, 93%, 94%, 95%, 96%, 97%, 98%, 99% or 100% sequence identity to the amino acid sequence set forth in SEQ ID NO:10.

In one embodiment, the invention provides a combination, as described herein, for use in the treatment of cancer, wherein the combination comprises an anti-BCMA antibody and daratumumab, wherein the anti-BCMA antibody comprises a CDRH1 comprising an amino acid sequence set forth in SEQ ID NO:1; a CDRH2 comprising an amino acid sequence set forth in SEQ ID NO:2; a CDRH3 comprising an amino acid sequence set forth in SEQ ID NO:3; a CDRL1 comprising an amino acid sequence set forth in SEQ ID NO:4; a CDRL2 comprising an amino acid sequence set forth in SEQ ID NO:5; and/or a CDRL3 comprising an amino acid sequence set forth in SEQ ID NO:6.

In one embodiment, the invention provides a combination, as described herein, for use in the treatment of cancer, wherein the combination comprises an anti-BCMA antibody and daratumumab, wherein the anti-BCMA antibody comprises a VH comprising an amino acid sequence set forth in SEQ ID NO:7; and/or a VL comprising an amino acid sequence set forth in SEQ ID NO:8.

In one embodiment, provided is the use of a combination in the manufacture of a medicament for use in the treatment of cancer. In another embodiment, provided is the use of a combination in the manufacture of a medicament for use in the treatment of cancer, wherein the combination comprises an anti-BCMA antigen binding protein and an immunomodulatory agent. In yet another embodiment, provided is the use of a combination in the manufacture of a medicament for use in the treatment of cancer, wherein the combination comprises an anti-BCMA antigen binding protein and an agent directed to ICOS. In yet another embodiment, provided is the use of a combination in the manufacture of a medicament for use in the treatment of cancer, wherein the combination comprises an anti-BCMA antigen binding protein and an anti-CD38 antigen binding protein.

### Treatment Schedules

The appropriate treatment schedule of the anti-BCMA antigen binding protein and the immunomodulatory agent will be determined readily by those of skill in the art.

In one exemplary treatment schedule, one dose of the anti-BCMA antigen binding protein is administered every 3 weeks (21-day cycle) for up to 16 cycles. In another exemplary treatment schedule, one dose of the anti-BCMA antigen binding protein is administered once weekly for three consecutive weeks followed by 1 week of rest (28-day cycle) for a maximum of 16 cycles. In yet another exemplary treatment schedule, one dose of anti-BCMA antigen binding protein is administered on day 1 of a 28-day cycle. In a further exemplary treatment schedule, one dose of anti-BCMA antigen binding protein is administered on day 1 of a 21-day cycle for up to 1 year.

In one exemplary treatment schedule, one dose of the agent directed to ICOS is administered every 3 weeks (21-day cycle) for up to 16 cycles. In another exemplary treatment schedule, one dose of the agent directed to ICOS is administered once weekly for three consecutive weeks followed by 1 week of rest (28-day cycle) for a maximum of 16 cycles. In yet another exemplary treatment schedule, one dose of the agent directed to ICOS is administered on day 1 of a 28-day cycle. In a further exemplary treatment schedule, one dose of the agent directed to ICOS is administered on day 1 of a 21-day cycle for up to 1 year.

In one exemplary embodiment, the immunomodulatory agent is daratumumab and the treatment schedule includes: a single dose of daratumumab each week during weeks 1-8, a single dose of daratumumab every two weeks during weeks 9-24, and a single dose of daratumumab every four weeks during weeks 25 and beyond.

In one exemplary embodiment, the immunomodulatory agent is daratumumab and the treatment schedule includes: a single dose of daratumumab each week during weeks 1-9, a single dose of daratumumab every three weeks during weeks 10-24, and a single dose of daratumumab every four weeks during weeks 25 and beyond.

### Kits

In some aspects, the disclosure provides a kit for use in the treatment of cancer comprising:
(i) an anti-BCMA antigen binding protein;
(ii) an agent directed to ICOS; and
(iii) instructions for use in the treatment of cancer.

In some embodiments, the anti-BCMA antigen binding protein and the agent directed to ICOS are each individually formulated in their own pharmaceutical compositions with one or more pharmaceutically acceptable carriers.

In some aspects, the disclosure provides a kit for use in the treatment of cancer comprising:
(i) an anti-BCMA antigen binding protein;
(ii) an anti-CD38 antigen binding protein; and
(iii) instructions for use in the treatment of cancer.

In some embodiments, the anti-BCMA antigen binding protein and the anti-CD38 antigen binding protein are each individually formulated in their own pharmaceutical compositions with one or more pharmaceutically acceptable carriers.

In some aspects, the disclosure provides a kit for use in the treatment of cancer comprising:
(i) an anti-BCMA antigen binding protein;
(ii) instructions for use in the treatment of cancer when combined with an agent directed to ICOS.

In some aspects, the disclosure provides a kit for use in the treatment of cancer comprising:
(i) an anti-BCMA antigen binding protein;
(ii) instructions for use in the treatment of cancer when combined with an anti-CD38 antigen binding protein.

SEQUENCE LISTINGS

SEQ. ID. NO. 1: anti-BCMA antibody CDRH1
NYWMH
SEQ. ID. NO. 2: anti-BCMA antibody CDRH2
ATYRGHSDTYYNQKFKG
SEQ. ID. NO. 3: anti-BCMA antibody CDRH3
GAIYDGYDVLDN
SEQ. ID. NO. 4: anti-BCMA antibody CDRL1
SASQDISNYLN
SEQ. ID. NO. 5: anti-BCMA antibody CDRL2
YTSNLHS
SEQ. ID. NO. 6: anti-BCMA antibody CDRL3
QQYRKLPWT
SEQ. ID. NO. 7: anti-BCMA antibody heavy chain
variable region
QVQLVQSGAEVKKPGSSVKVSCKASGGTFSNYWMIHWVRQAPGQGLEWMG
ATYRGHSDTYYNQKFKGRVTITADKSTSTAYMELSSLRSEDTAVYYCARG
AIYDGYDVLDNWGQGTLVTVSS
SED. ID. NO. 8: anti-BCMA antibody light chain
variable region
DIQMTQSPSSLSASVGDRVTITCSASQDISNYLNWYQQKPGKAPKLLIYY
TSNLHSGVPSRFSGSGSGTDFTLTISSLQPEDFATYYCQQYRKLPWTFGQ
GTKLEIKR
SEQ. ID. NO. 9: anti-BCMA antibody heavy chain
region
QVQLVQSGAEVKKPGSSVKVSCKASGGTFSNYWMHWVRQAPGQGLEWMGA
TYRGHSDTYYNQKFKGRVTITADKSTSTAYMELSSLRSEDTAVYYCARGA
IYDGYDVLDNWGQGTLVTVSSASTKGPSVFPLAPSSKSTSGGTAALGCLV
KDYFPEPVTVSWNSGALTSGVHTFPAVLQSSGLYSLSSVVTVPSSSLGTQ
TYICNVNHKPSNTKVDKKVEPKSCDKTHTCPPCPAPELLGGPSVFLFPPK
PKDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPREP
QVYTLPPSRDELTKNQVSLTCLVKGFYPSDIAVEWESNGQPENNYKTTPP
VLDSDGSFFLYSKLTVDKSRWQQGNVFSCSVMHEALHNHYTQKSLSLSPG
K
SEQ. ID. NO. 10: anti-BCMA antibody light chain
region
DIQMTQSPSSLSASVGDRVTITCSASQDISNYLNWYQQKPGKAPKLLIYY
TSNLHSGVPSRFSGSGSGTDFTLTISSLQPEDFATYYCQQYRKLPWTFGQ
GTKLEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC
SEQ ID. NO. 11: human ICOS (isoform 2)(Accession
No.: UniProtKB - Q9Y6W8-2)
MKSGLWYFFLFCLRIKVLTGEINGSANYEMFIFHNGGVQILCKYPDIVQQ
FKMQLLKGGQILCDLTKTKGSGNTVSIKSLKFCHSQLSNNSVSFFLYNLD
HSHANYYFCNLSIFDPPPFKVTLTGGYLHIYESQLCCQLKFWLPIGCAAF
VVVCILGCILICWLTKKM
SEQ ID. NO. 12: human ICOS (isoform 1)(Accession
No.: UniProtKB - Q9Y6W8-1)
MKSGLWYFFLFCLRIKVLTGEINGSANYEMFIFHNGGVQILCKYPDIVQQ
FKMQLLKGGQILCDLTKTKGSGNTVSIKSLKFCHSQLSNNSVSFFLYNLD
HSHANYYFCNLSIFDPPPFKVTLTGGYLHIYESQLCCQLKFWLPIGCAAF
VVVCILGCILICWLTKKKYSSSVHDPNGEYMFMRAVNTAKKSRLTDVTL
SEQ. ID. NO. 13: anti-ICOS antibody CDRH1
DYAMH
SEQ. ID. NO. 14: anti-ICOS antibody CDRH2
LISIYSDHTNYNQKFQG
SEQ. ID. NO. 15: anti-ICOS antibody CDRH3
NNYGNYGWYFDV
SEQ. ID. NO. 16: anti-ICOS antibody CDRL1
SASSSVSYMH
SEQ. ID. NO. 17: anti-ICOS antibody CDRL2
DTSKLAS
SEQ. ID. NO. 18: anti-ICOS antibody CDRL3
FQGSGYPYT
SEQ. ID. NO. 19: anti-ICOS antibody heavy chain
variable region (H2)
QVQLVQSGAEVKKPGSSVKVSCKASGYTFTDYAMHWVRQAPGQGLEWMGL
ISIYSDHTNYNQKFQGRVTITADKSTSTAYMELSSLRSEDTAVYYCGRNN
YGNYGWYFDVWGQGTTVTVSS
SEQ. ID. NO. 20: anti-ICOS antibody light chain
variable region (L5)
EIVLTQSPATLSLSPGERATLSCSASSSVSYMHWYQQKPGQAPRLLIYDT
SKLASGIPARFSGSGSGTDYTLTISSLEPEDFAVYYCFQGSGYPYTFGQG
TKLEIK
SEQ. ID. NO. 21: 37A10S713 heavy chain variable
region
EVQLVESGG LVQPGGSLRL SCAASGFTFS DYWMDWVRQA
PGKGLVWVSN IDEDGSITEY SPFVKGRFTI SRDNAKNTLY
LQMNSLRAED TAVYYCTRWG RFGFDSWGQG TLVTVSS
SEQ. ID. NO. 22: 37A10S713 light chain variable
region
DIVMTQSPDS LAVSLGERAT INCKSSQSLL SGSFNYLTWY
QQKPGQPPKL LIFYASTRHT GVPDRFSGSG SGTDFTLTIS
SLQAEDVAVY YCHHHYNAPP TFGPGTKVDI K
SEQ. ID. NO. 23: 37A10S713 VH CDR1
GFTFSDYWMD
SEQ. ID. NO. 24: 37A10S713 VH CDR2
NIDEDGSITEYSPFVKG
SEQ. ID. NO. 25: 37A10S713 VH CDR3
WGRFGFDS
SEQ. ID. NO. 26: 37A10S713 VL CDR1:
KSSQSLLSGSFNYLT
SEQ. ID. NO. 27: 37A10S713 VL CDR2
YASTRHT
SEQ. ID. NO. 28: 37A10S713 VL CDR3
HHHYNAPPT

### EXAMPLES

### Example 1

### In vivo Study of an Anti-BCMA Antibody Drug Conjugate in Combination With an Agent Directed to ICOS.

### 1.1 Animals

Female C57BL/6 mice from Charles River were used in this study. The mice were housed under conditions outlined in the NIH Guide for Care and Use of Laboratory Animals in compliance with the USDA Laboratory Animal Welfare Act, in a fully accredited AAALAC facility.

### 1.2 Cell Culture

EL4 mouse lymphoma cells overexpressing human BCMA (EL4-hBCMA) were generated. EL4-hBCMA were removed from liquid nitrogen storage, thawed and transferred to a culture flask containing sterile media (DMEM, containing 10% fetal bovine serum (FBS)). The cells were sub-cultured a minimum of three times prior to inoculation. To select BCMA expressing cells, G418 (final concentration 0.2 mg/ml in media) was added at the first passage and every subsequent passage. Tumor cells were maintained in tissue culture flasks in a humidified incubator at 370C in 5% CO2. Immediately prior to inoculation cells were washed three times with ice cold DMEM and brought to a concentration of 1.0×106 cells/ml in cold DMEM. Inoculation stocks were placed on ice and transferred to vivarium for injection.

### 1.3 C57BL/6 Inoculation

C57BL/6 female mice were implanted with an identification chip (BMDS IMI-1000 transponder) prior to cell inoculation. EL4-hBCMA cells harvested during exponential growth, were injected into the right flank of each mouse (1.0×105 cells in 0.1 mLs).

### 1.4 Randomization and Treatment

Mice were monitored for noticeable tumor growth and measured when palpable tumors were observed. On day eleven after inoculation, tumor volumes for each mouse were recorded. Mice were grouped using StudyLog Study Director Suite randomization function using matched distribution. In this study, day zero was randomization and day of initial treatment.

### 1.6 Tumor Measurement

Tumor growth was measured using a Fowler “ProMax” digital calliper. Length and width of tumors were measured in order to determine tumor volume using the formula (tumor volume=0.52×L×W2). Tumor volume data was recorded using Studylog Study Director Suite software. Tumor measurements of greater than 2,000 mm3 for an individual mouse resulted in the mouse being removed from study and euthanization.

### 1.7 Experimental Protocol

The purpose of this experiment was to evaluate the anti-tumor activity of an anti-BMCA antigen binding protein in combination with an agent directed to ICOS in the EL4-hBCMA mouse syngeneic tumor model. Mice were randomized into the following four (4) groups when the average tumor volume reached 150-200 mm3:
IgG1/MMAF
IgG1/GSK2857916
ICOS/MMAF
ICOS/GSK2857916

“GSK2857916” (or “GSK916”) is an anti-BCMA IgG1 monoclonal antibody conjugated to the cytotoxin MMAF comprising a heavy chain variable region (VH) comprising an amino acid sequence as set forth in SEQ ID NO:7 and a light chain variable region (VL) comprising an amino acid as set forth in SEQ ID NO:8, and was dose at a concentration of 10 mg/kg.

“ICOS” is an anti-mouse IgG1 monoclonal antibody (clone 7E.17G9, expressed on a mouse IgG1 isotype) and was dosed at a concentration of 200 μg/mouse.

The “MMAF” control group contains the cytotoxin MMAF conjugated to a human IgG1 and is a control for GSK2857916.

The “IgG1” control group is an isotype control for the ICOS antibody.

All compounds were administered two times a week over three weeks.

### 1.8 Statistical Analysis

All statistical analysis was done with R software.

### 1.9 Results

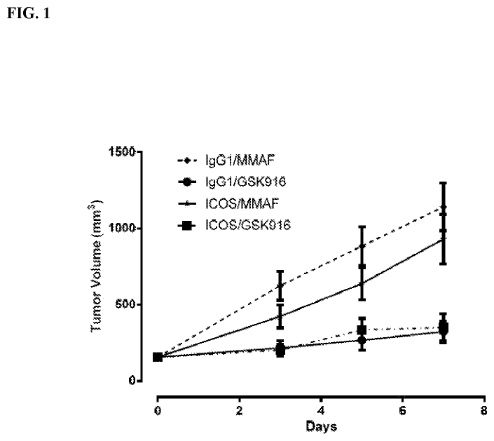

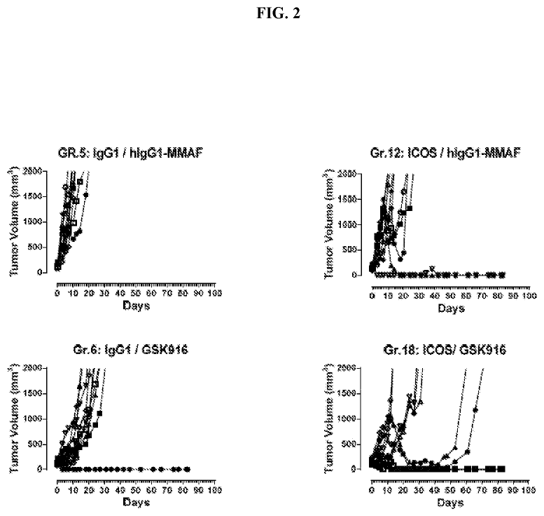

FIG. 1 shows the mean tumor volume data on day 7. FIG. 2 shows the individual tumor volume curves for groups treated for up to 83 days. A similar tumor growth inhibition was observed with GSK2857916 monotherapy (IgG1/GSK2857916) and with ICOS in combination GSK2857916 (ICOS/GSK2857916). Complete tumor regressions were observed by study day 83. ICOS monotherapy resulted in 20% tumor free mice, and GSK2857916 resulted in 10% tumor free mice. The combination of ICOS plus GSK2857916 resulted in 30% tumor free mice. Thus, this combination showed a trend of 10%-20% survival advantage over ICOS or GSK2857916 alone.

1. A method of treating cancer in a subject in need thereof comprising administering a therapeutically effective dose of a combination comprising an anti-BCMA antigen binding protein and an agent directed to ICOS.

2. A method of treating cancer in a subject in need thereof comprising administering a therapeutically effective dose of a combination comprising an anti-BCMA antigen binding protein and an anti-CD38 antigen binding protein.

3. The method of claim 1, wherein the wherein the anti-BCMA antigen binding protein comprises a CDRH1 comprising an amino acid sequence with at least 90% sequence identity to the amino acid sequence set forth in SEQ ID NO:1; a CDRH2 comprising an amino acid sequence with at least 90% sequence identity to the amino acid sequence set forth in SEQ ID NO:2; a CDRH3 comprising an amino acid sequence with at least 90% sequence identity to the amino acid sequence set forth in SEQ ID NO:3; a CDRL1 comprising an amino acid sequence with at least 90% sequence identity to the amino acid sequence set forth in SEQ ID NO:4; a CDRL2 comprising an amino acid sequence with at least 90% sequence identity to the amino acid sequence set forth in SEQ ID NO:5; and a CDRL3 comprising an amino acid sequence with at least 90% sequence identity to the amino acid sequence set forth in SEQ ID NO:6.

4. The method of claim 1, wherein the wherein the anti-BCMA antigen binding protein is an antibody comprising a heavy chain variable region (VH) comprising an amino acid sequence with at least 90% sequence identity to the amino acid sequence set forth in SEQ ID NO:7; and a light chain variable region (VL) comprising an amino acid sequence with at least 90% sequence identity to the amino acid sequence set forth in SEQ ID NO:8.

5. The method of claim 1, wherein the anti-BCMA antigen binding protein is an immunoconjugate comprising an antibody conjugated to a cytotoxin.

6. The method of claim 5, wherein the cytotoxin is selected from MMAE or MMAF.

7. The method of claim 1, wherein the agent directed to ICOS is an anti-ICOS antibody.

8. The method of claim 7, wherein the anti-ICOS antibody is an ICOS agonist.

9. The method of claim 7, wherein the anti-ICOS antibody comprises a CDRH1 comprising an amino acid sequence with at least 90% sequence identity to the amino acid sequence set forth in SEQ ID NO:13; a CDRH2 comprising an amino acid sequence with at least 90% sequence identity to the amino acid sequence set forth in SEQ ID NO:14;
a CDRH3 comprising an amino acid sequence with at least 90% sequence identity to the amino acid sequence set forth in SEQ ID NO:15; a CDRL1 comprising an amino acid sequence with at least 90% sequence identity to the amino acid sequence set forth in SEQ ID NO:16; a CDRL2 comprising an amino acid sequence with at least 90% sequence identity to the amino acid sequence set forth in SEQ ID NO:17; and a CDRL3 comprising an amino acid sequence with at least 90% sequence identity to the amino acid sequence set forth in SEQ ID NO:18.

10. The method of claim 7, wherein the anti-ICOS antibody comprises a VH domain comprising an amino acid sequence at least 90% identical to the amino acid sequence set forth in SEQ ID NO:19; and a VL domain comprising an amino acid sequence at least 90% identical to the amino acid sequence as set forth in SEQ ID NO:20.

11. The method of claim 1 wherein the agent directed to ICOS comprises an Fc region comprising a S228P mutation and L235E mutation.

12. The method of claim 2 wherein anti-CD38 antigen binding protein is an anti-CD38 antibody.

13. The method of claim 12, wherein the ant-CD38 antibody is daratumumab.

14. The method of claim 1, wherein the cancer is selected from multiple myeloma, chronic lymphocytic leukemia, Waldenstroem's Macroglobulinemia, and non-Hodgkin's lymphoma.

15. (canceled)

16. (canceled)

17. (canceled)

18. (canceled)

19. A kit for use in the treatment of cancer comprising:
(i) an anti-BCMA antigen binding protein;
(ii) instructions for use in the treatment of cancer when combined with an agent directed to ICOS.

20. A kit for use in the treatment of cancer comprising:
(i) an anti-BCMA antigen binding protein;
(ii) instructions for use in the treatment of cancer when combined with an anti-CD38 antigen binding protein.

21. The method of claim 1, wherein 1.9 mg/kg, 2.5 mg/kg, or 3.4 mg/kg of an anti-BCMA antigen binding protein is administered on day 1 of a 21-day cycle.

22. The method of claim 1, wherein 8 mg, 24 mg, or 80 mg of an agent directed to ICOS is administered every three weeks.

23. A method of treating relapsed/refractory multiple myeloma in a subject that has been treated with at least three prior cancer treatments comprising administering:
1.9 mg/kg, 2.5 mg/kg, or 3.4 mg/kg of an anti-BCMA antibody-drug conjugate on day 1 of a 21-day cycle; wherein the anti-BCMA antibody drug conjugate comprises an antibody comprising the heavy chain amino acid sequence set forth in SEQ ID NO:9 and the light chain amino acid sequence set forth in SEQ ID NO:10, and wherein the antibody is conjugated to MMAF; and
8 mg, 24 mg, or 80 mg of an anti-ICOS antibody every three weeks; wherein the anti-ICOS antibody comprises a VH domain comprising the amino acid sequence set forth in SEQ ID NO:19; and a VL domain comprising the amino acid sequence set forth in SEQ ID NO:20.

24. A method of treating primary amyloidosis (AL) in a subject in need thereof comprising administering a therapeutically effective dose of a combination comprising an anti-BCMA antigen binding protein and an agent directed to ICOS.
